(19) United States
(12) Patent Application Publication
(10) Pub. No.: US 2021/0270815 A1
(43) Pub. Date: Sep. 2, 2021

(54) SPERM FERTILITY CAPACITY TEST AND SPERM DECAPACITATING SUPPLEMENT

(71) Applicants: PETER SUTOVSKY, COLUMBIA, MO (US); MICHAL ZIGO, COLUMBIA, MO (US); KARL KERNS, ASHLAND, MO (US); MIRIAM SUTOVSKY, COLUMBIA, MO (US)

(72) Inventors: PETER SUTOVSKY, COLUMBIA, MO (US); MICHAL ZIGO, COLUMBIA, MO (US); KARL KERNS, ASHLAND, MO (US); MIRIAM SUTOVSKY, COLUMBIA, MO (US)

(21) Appl. No.: 17/056,446

(22) PCT Filed: May 17, 2019
(86) PCT No.: PCT/US2019/032860

§ 371 Date: Nov. 18, 2020

Related U.S. Application Data
(60) Provisional application No. 62/673,346, filed on May 18, 2018.

(51) Int. Cl. G01N 33/50

(52) U.S. Cl. CPC G01N 33/5091; G01N 2800/344

(57) ABSTRACT

Disclosed herein are methods for determining the fertility of a sperm sample or a source of a sperm sample; the methods comprising labeling the sperm sample with a zinc probe, identifying presence and/or localization of zinc associated with spermatozoa in the sample and determining the fertility of the sample based on the zinc presence and/or localization in the spermatozoa as compared to a reference pattern associated with non-capacitated spermatozoa or a reference pattern associated with sperm capacitation. Also disclosed are methods of improving sperm fertility and/or decreasing premature capacitation while handling, storing, or transporting semen, the methods comprising adding exogenous zinc to a sperm sample or semen sample. Also disclosed are compositions for improving sperm fertility and/or reducing premature capacitation, the compositions comprising exogenous zinc ions.

### CROSS-REFERENCE TO RELATED APPLICATIONS

This application is based on and claims priority to U.S. Provisional Application No. 62/673,346 filed on May 18, 2018, which is hereby incorporated herein by reference. In its entirety.

### FIELD OF THE INVENTION

The present invention relates to methods and compositions for determining sperm fertility, improving sperm fertility and preserving sperm and/or semen for use in artificial reproductive technologies.

### BACKGROUND OF THE INVENTION

Mammalian spermatozoa are unique in that they are deposited in the female reproductive tract in an immature state. In order to successfully fertilize an oocyte, spermatozoa must undergo capacitation. This process encompasses an influx of bicarbonate and calcium ions, removal of decapacitating factors, changes of intracellular pH and sperm proteasomal activities. Sperm that have undergone capacitation exhibit hyperactivity and have disrupted acrosomal membranes to allow for penetration of the zona pellucida of the female oocyte and successful fertilization. However, although sperm capacitation is required for fertility, it is a terminal maturation event that leads to rapid cell death unless fertilization occurs. Therefore, it is necessary that capacitation occur at an optimal time. Premature capacitation can lead to reduced fertility and quality of a semen sample.

What is needed is an accurate and effective way to identify sperm samples having high fertility (e.g., having the maximal potential for successful capacitation near an oocyte) and to optimize or improve sperm fertility. Further, methods to prevent premature or mistimed capacitation are needed to improve the quality of sperm during transport or storage of semen (e.g., for artificial reproduction techniques).

### BRIEF SUMMARY OF THE INVENTION

Provided herein are methods for determining fertility of spermatozoa in a sperm sample obtained from a sperm source or for determining fertility of the sperm source, the methods comprising: labeling the sperm source with a zinc probe; identifying presence and/or localization of zinc associated with the spermatozoa in the sample; and comparing the presence and/or localization of the zinc associated with the spermatozoa to a reference pattern of zinc presence and/or localization associated with sperm capacitation to determine whether or not the spermatozoa in the sample or the source of the sperm sample is fertile.

Also provided are methods for improving the fertility of spermatozoa in a sperm sample, the method comprising adding exogenous zinc ions to the sperm sample.

Also provided are kits for determining the fertility of spermatozoa comprising: a zinc probe and one or more of (a) a compound for detecting plasma membrane integrity, (b) a compound for detecting modifications to the acrosome, and (c) a DNA dye.

Further provided are compositions for preventing premature sperm capacitation and methods of using thereof, the compositions comprising exogenous zinc ions.

Other objects and features will be in part apparent and in part pointed out hereinafter.

### BRIEF DESCRIPTION OF THE DRAWINGS

FIG. 1A shows representative epifluorescence (white) of porcine spermatozoa having zinc signatures 1-4. Scale bar is 25 μm.

FIG. 1B shows representative epifluorescence (white) of bovine spermatozoa having zinc signatures 1-4. Scale bar is 25 μm.

FIG. 1C shows representative epifluorescence (white) human spermatozoa having zinc signatures 1-4. Scale bar is 20 μm.

FIG. 1D shows boar spermatozoa after 72 hours of storage in Beltsville Thaw Solution (BTS semen extender) under epifluorescence showing varied zinc signatures. Scale bar is 25 μm.

FIG. 2A illustrates the gating approach for measuring sperm zinc signatures. The left panel demonstrates how initial events collected from FlowSight data acquisition were gated for cell in focus as a function of brighfield gradient RMS (a calculation of image crispness). The middle panel shows how events in focus are further gated to analyze only single spermatozoa, plotted as H33342 fluorescence area by aspect ratio. The right panel identifies differentiated and gated out laterally aligned spermatozoa by using a plot of brightfield standard deviation by brightfield H entropy mean.

FIG. 2B indicates a masking approach where H33342 fluorescence (middle panel on the left set of panels) is subtracted from the normal brightfield image (first panel on the left set of panels), to generate the resulting image of the tail alone (last panel on the left set of panels). The right set of panels shows representative images of each of the four zinc signatures indicating zinc signals in the tail only vs. zinc and nuclei staining.

FIG. 2C shows a combined gating and masking analysis of spermatozoa. The left panel shows F73 fluorescence of all cells. The right panel shows single, focused, aligned sperm cells with the FZ3 tail fluorescence only.

FIG. 3 shows an anti-phosphotyrosine Western blot of sperm extracts from various treatments (Left panel) and a corresponding loading control anti-tubulin Western blot (Right panel) with quantification for normalization. The following sperm extract conditions were loaded in the following lanes in the left panel: 1) marker; 2) ejaculated, non-IVC; 3) experimental IVC conditions (2 mM sodium bicarbonate, 5 mM pyruvate); comparison IVC (15 mM sodium bicarbonate, 0.2 mM pyruvate); 5) comparison IVC (15 mM sodium bicarbonate, 5 mM pyruvate).

FIG. 4A shows time lapse recordings of zinc signature during IVC in high, 15 mM sodium bicarbonate media. The left panel shows plasma membrane changes as identified by PI status, distinguishing between IVC-induced PI+ subpopulations (PI+ live with plasma membrane changes vs. PI+ cell death). The right panel shows acrosomal modifications.

FIG. 4B shows time lapse recordings of zinc signature during IVC in low (2 mM) sodium bicarbonate media. Left panel shows plasma membrane changes and right panel shows acrosomal modifications (corresponding histogram color code for time points in figure legend).

FIG. 5A depicts lectin peanut agglutin (PNA) status by Zn signature. PNA indicates acrosomal status. Zinc signatures were divided into three groups (panels below) indicating no remodeling, acrosome exocytosed or acrosome remodeled.

FIG. 5B illustrates PI plasma membrane integrity across zinc signatures (listed above). Zinc signature status corresponds with PI plasma membrane integrity in fresh (dark gray) and IVC spermatozoa (light gray).

FIG. 5C illustrates acrosome status by membrane intensity. PNA intensity is plotted along the x-axis and acrosome status is indicated. As sperm plasma membrane-integrity decreased, acrosomal remodeling and exocytosis occurred. P-values determined by the General Linear Model procedure in SAS 9.4

FIG. 6A shows the zinc signatures of human spermatozoa after IVC. Scale bar 20 um.

FIG. 6B shows a time lapse Zn signature cytometry during in vitro capacitation every 30 minutes between 0-2 hours, then every hour until 6 hours, gray scaled code of time points located in figure legend.

FIG. 7A shows zinc signature histograms as determined from IBFC analysis of sperm obtained or stored in various conditions: fresh/ejaculated, incubated/Non-IVC, 100 uM MG132+IVC, IVC+Vehicle, 1 mM ZnCl₂+IVC, Zn-chelator, 10 uM TPEN).

FIG. 7B shows pie charts depicting the zinc signature distributions in select IVC treatments. Treatment P-values were determined by the General Linear Model procedure in SAS 9.4. 10,000 sperm per sample analyzed.

FIG. 7C shows zinc signature histograms of non-capacitated (non-IVC, white) and capacitated (IVC, red) sperm populations from IBFC analysis (top panel) and then representative images from IBFX analysis showing each of the individual zinc signatures (bottom panels, scale bar: 20 μm). Each spermatozoon in the bottom panels has the following images acquired: bright field (BF); Zn²⁺ reporting probe FZ3 (Zn), sperm viability/plasma membrane integrity probe propidium iodide (PI); live DNA stain Hoechst 33342 (DNA); and side scatter (not displayed), with a merger of the four images (Merge).

FIG. 8A show representative zinc signature histograms from a second biological replicate of IVC proteasomal inhibitor treatments.

FIG. 8B show a TPEN F73 zinc probe vehicle treatment. Vehicle did not shift zinc signature compared to no vehicle or TPEN treatment.

FIG. 8C shows representative zinc signature histograms during sequential extraction of fresh, ejaculated spermatozoa (e.g., during PBS wash (left panel), increasing the ionic strength to 0.75 M KCL in PBS (middle panel) and at 30 mM nonionic detergent (OBG) in PBS (right panel)).

FIG. 9 shows zinc signature associated with varied fertility in AI boars. Left graph shows the zinc signature in four boars of known high or low fertility, before and after IVC. Right graphs show the IVC induced fold increase in Signature 3 among high and low fertility boars.

FIG. 10 shows zinc signature histograms of four bars with known high or low fertility, before and after IVC.

FIG. 11A indicates an interpretation of zinc signature meaning and population segregation: 16% of fresh, ejaculated spermatozoa had undergone early stage capacitation upon semen collection (lightest gray working to darkest); 14% of spermatozoa spontaneously undergo early stage capacitation during incubation without IVC-inducers; 60% of spermatozoa remained capacitation competent with IVC-inducers, with 21% sensitive to proteasomal inhibition; remaining 10% of sperm were capacitation incompetent under IVC-conditions (darkest gray) (standard error bars included).

FIG. 11B diagrams proposed zinc signature changes throughout female reproductive tract and oocyte zinc spark interference with sperm zinc signature as a combined polyspermy defense mechanism, the zinc shield.

Corresponding reference characters indicate corresponding parts throughout the drawings.

### DETAILED DESCRIPTION OF PREFERRED EMBODIMENT

Provided herein are methods of identifying and using fertile sperm samples according to novel zinc signatures found to be associated with capacitation and fertility. The methods described herein provide an efficient method of identifying fertile sperm samples to allow for greater success in artificial reproductive technologies. A method of preserving sperm/semen from premature maturation and subsequent degradation is also provided. The methods and compositions provided herein may be used to improve the efficiency and proficiency of artificial reproductive technologies.

Preferred methods and materials are described below, although methods and material similar or equivalent to those described herein can be used in practice or testing of the present invention. All publications, patent applications, patents and other references mentioned herein are incorporated by reference in their entirety. The materials, methods and examples disclosed herein are illustrative only and not intended to be limiting.

### Definitions

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, cell imaging, reproductive biology, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

As used herein, the term “acrosome” refers to an organelle that develops over the anterior half of the head in the spermatozoa. It plays a role in the acrosomal reaction (i.e., acrosomal exocytosis) required for successful fertilization where the membrane surrounding the acrosome fuses with the plasma membrane of the sperm's head, exposing the contents of the acrosome. These contents include surface antigens necessary for binding to the cell membrane of the egg (ovum) as well as numerous enzymes that breakdown the coating that surrounds the egg (ovum). As used herein, “acrosomal modification” refers to any modification or changes that occur to the acrosome to prepare for, or during, this process.

“DNA dye” and a “DNA stain” are used herein to refer to substances that bind to and label DNA. As used herein, a DNA dye is capable of permeating a living cell membrane and can therefore stain the nuclei of living, intact cells comprising an uncompromised plasma membrane. As used herein, a DNA stain is incapable of permeating uncompromised/intact plasma membranes and so preferably labels the nuclei of cells comprising compromised or disrupted plasma membranes. Exemplary DNA dyes and DNA stains are described herein below. Both the DNA dyes and DNA stains herein may comprise fluorescent or visible markers.

As used herein the term “breeding” encompasses any form of natural and artificial reproduction. Breeding can occur naturally or may be induced by artificial means. For example, it can occur via artificial insemination or in vitro fertilization.

As used herein the term “fertile” or “fertility” refers to the ability of a spermatozoa to fertilize an egg. In various embodiments, fertility can refer to the ability to penetrate the zona pellucida, an ability to achieve sperm oocyte activation, an ability to progress an oocyte or zygote out of metaphase II and/or an ability to achieve a blastocyst stage. Generally, a fertile sample is capable of conceiving young (either in vitro or in vivo).

As used herein, the term “infertile” refers to an inability to conceive young, an inability to fertilize an egg, an inability to penetrate the zona pellucida, an inability to achieve sperm oocyte activation, an inability to progress an oocyte or zygote out of metaphase II and/or an inability to achieve a blastocyst stage.

As used herein, the term “sub-fertile” refers to a reduced ability to conceive young, a reduced ability to fertilize an egg, a reduced ability to penetrate the zona pellucida, a reduced ability to achieve sperm oocyte activation, a reduced ability to progress an oocyte or zygote out of metaphase II and/or a reduced ability to achieve a blastocyst stage compared to a fertile sample.

### I. Methods of Determining Sperm Fertility

Provided herein are methods for determining sperm fertility. The methods comprise labeling a sperm sample with a zinc probe and identifying presence and/or localization of zinc associated with the spermatozoa in the sample. Advantageously, the spermatozoa may be identified as fertile or infertile depending on the presence and/or localization of zinc in the sample.

Therefore, in various embodiments a method for determining sperm fertility is provided, the method comprising labeling a sperm sample with a zinc probe and comparing presence and/or localization of zinc associated with the spermatozoa in the sample with a reference pattern of presence and/or localization of zinc associated with sperm capacitation. The spermatozoa may be identified as fertile or infertile depending on the localization and/or presence of zinc associated with it (i.e., its zinc signature profile).

In various embodiments, the sperm sample is determined to comprise fertile spermatozoa when spermatozoa are identified having a zinc signature 1 and/or a zinc signature 2.

In various embodiments, the zinc signature 1 is characterized by zinc presence and/or localization in the sperm head and whole tail. The zinc signature 1 is generally associated with non-capacitated spermatozoa.

In various embodiments, the zinc signature 2 is characterized by zinc presence and/or localization in both the sperm head and tail midpiece, excluding the tail principal piece. The zinc signature 2 is generally associated with hyperactivated spermatozoa capable of recognizing and binding an oocyte zona pellucida.

Alternatively, or in addition, the sperm sample may be determined to comprise infertile spermatozoa when spermatozoa are identified having zinc signature 3 and/or zinc signature 4.

In various embodiments, the zinc signature 3 is characterized by localization of zinc only in the sperm tail midpiece. The zinc signature 3 is generally associated with spermatozoa that are undergoing or have fully underwent capacitation, have undergone acrosomal modification and/or have a compromised and/or remodeled plasma membrane.

In various embodiments, the zinc signature 4 is characterized by essentially no zinc presence in the spermatozoa. As used herein, “essentially no zinc presence” may allow for a small, residual amount of zinc. However it should be understood that any zinc associated with spermatozoa in zinc signature 4 is significantly less than any zinc identified in any of the other three zinc signatures. In various embodiments, the zinc signature 4 is characterized by no zinc presence in the spermatozoa. The zinc signature 4 is generally associated with spermatozoa having a compromised and/or remodeled plasma membrane and/or an exocytosed acrosome. In various embodiments, spermatozoa having zinc signature 4 may be undergoing or have already undergone cell death (i.e., following capacitation).

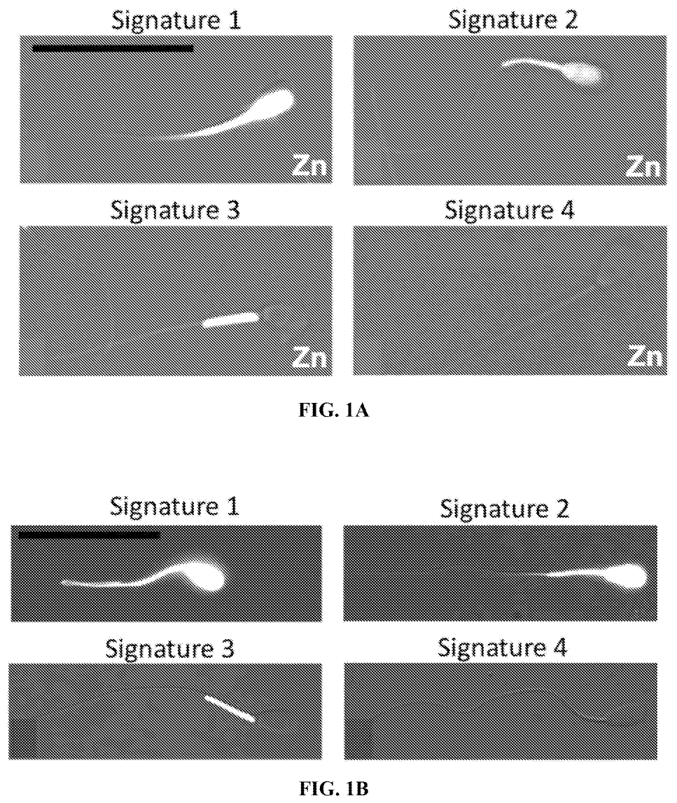

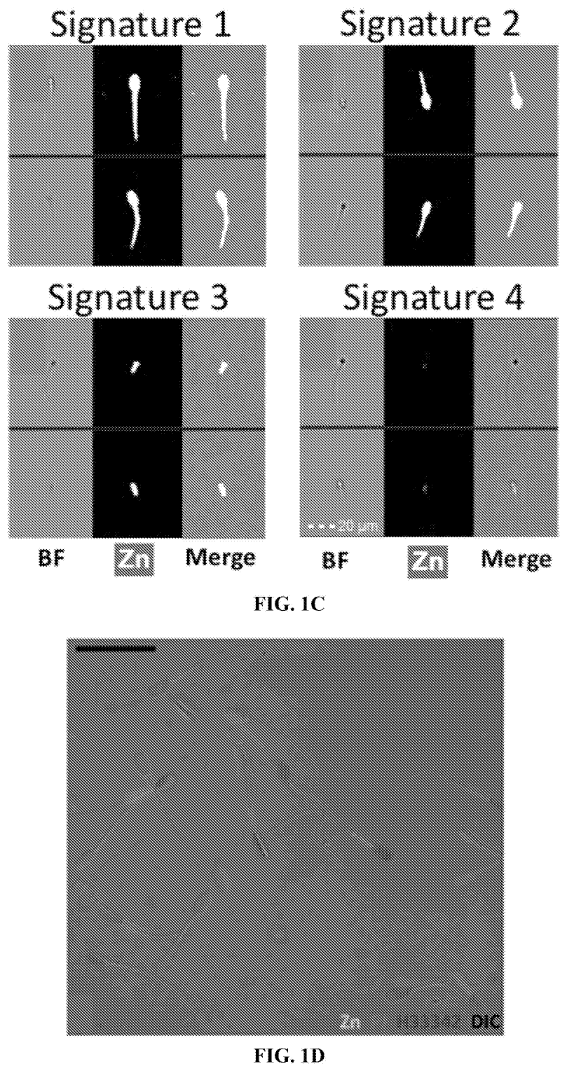

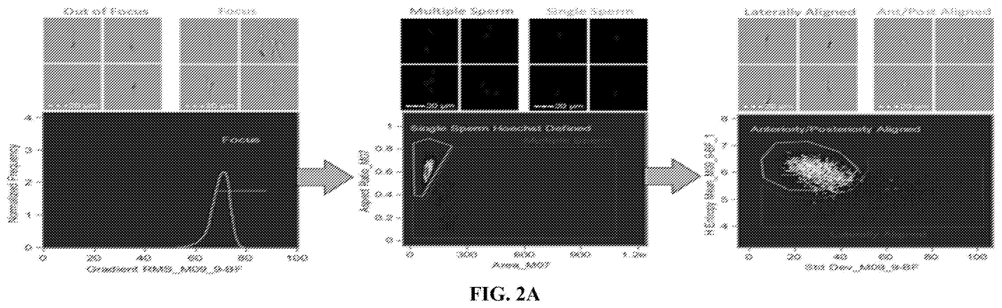

Representative examples of epifluorescence associated with signatures 1-4 are shown in FIG. 1A (porcine), FIG. 1B (bovine), an FIG. 1C (human), as described in more detail in Example 1, below.

In various embodiments, the zinc signature may present as an intermediate between any two of the zinc signatures described herein. For example, in some embodiments, the spermatozoa may present with a zinc signature wherein the zinc is localized to the sperm acrosome or the acrosome and sperm midpiece. This signature can exist momentarily between the zinc signature 2 and the zinc signature 3 described herein. In various embodiments, spermatozoa presenting with this intermediate sperm signature may be classified as having declining fertility.

Any zinc probe may be used in the methods described herein. Suitable zinc probes are provided by Santa Cruz Biotechnology (e.g., TFL-Zn potassium salt, ZnAF-1, ZnAF-1F, ZnAF-2F, ethyl 2-(2-isobutyl-6-quinolyloxy-8-p-toluenesulfonamido)acetate, (alphaS)-5-[(Dimethylamino)sulfonyl]-alpha-[[(9H-fluoren-9-ylmethoxy)carbonyl]amino]-8-hydroxy-2-quinolinepropanoic Acid, Zinquin ethyl ester, ZnAF-2 tetrahydrochloride, Ethyl 2-(2-[(E)-2-Phenyl-1-ethenyl]-6-quinolyloxy-8-p-toluenesulfonamido)acetate, Ethyl 2-[2-(2-Methyl-1-propenyl)-6-quinolyloxy-8-p-toluenesulfonamido)acetate, and 5-Bromo-PAPS), Sigma Aldrich (e.g., N1-(7-Nitro-2,1,3-benzoxadiazol-4-yl)-N1,N2,N2-tris(2-pyridinylmethyl)-1,2-ethanediamine, ZnAF-2 DA, and ZnAF-1 DA) or by Thermofisher Scientific (FLUOZIN-1 tripotassium salt, FLUOZIN-1 AM, FLUOZIN-3-AM, FLUOZIN-3 tetrapotassium salt). In various embodiments, the zinc probe comprises FLUOZIN-3 AM (FZ3).

In further embodiments, the methods for determining sperm fertility may further comprise labeling the sperm sample with a compound for detecting a disrupted plasma membrane and/or a compound for detecting modifications to an acrosome; and determining plasma membrane disruption and/or acrosomal modifications in the sample. In various embodiments, the sperm sample is labeled with these compound(s) independently from the application and evaluation of the zinc probe to the same sample as described above. In various embodiments, more than one sperm sample may be obtained from a single sperm source and each independently evaluated for zinc presence and/or localization, plasma membrane disruptions and/or acrosomal modifications to determine the overall fertility of the sperm source. Thus, in various embodiments, one or more sperm samples obtained from a sperm source may each be independently labeled with (a) a zinc probe, (b) a compound for detecting a disrupted plasma membrane and/or (c) a compound for detecting modifications to an acrosome to determine the fertility of the one or more sperm samples (and by extension, the sperm source). Alternatively, the same sperm sample may be labeled with the zinc probe and a compound for detecting a disrupted plasma membrane and/or a compound for detecting modifications to an acrosome. The labeling with the compounds for detecting a disrupted plasma membrane and/or acrosomal modifications may occur at any point in relation to the application and evaluation of the zinc probe.

Any compound that can detect a disrupted or compromised plasma membrane may be used in the methods described herein. In various embodiments, the compound can comprise a DNA stain that can label cell nuclei, but is normally excluded from the cell when faced with an intact plasma membrane. Thus, in various embodiments, plasma membrane disruption may be detected using this DNA stain when nuclei of the spermatozoa are visible. Suitable DNA stains can include, for example, propidium iodide. DNA stains may be visible stains and/or may emit fluorescence.

Any compound known in the art for detecting acrosomal modifications may be used in the methods described herein. In various embodiments, the compound for detecting modification in the acrosome may bind to the outer acrosomal membrane of compromised and/or modified sperm acrosomes but not to acrosomal membranes in intact acrosomes. Suitable acrosomal labeling compounds can comprise labeled lectins. The lectin can comprise concanavalin A, Datura stramonium Lectin, Dolichos biflorus Agglutinin, Griffonia simplicifolia Lectin I, Griffonia simplicifolia Lectin II, Lectin isolated from Artocarpus integrifolia (i.e., Lens culinaris (Lentil) Agglutinin), Lycopersicon esculentum (Tomato) Lectin, Phaseolus vulgaris Erythroagglutinin, Peanut agglutinin, Pisum sativum Agglutinin, Ricinus communis Agglutinin 1, Soybean Agglutinin, Ulex europaeus Agglutinin I, Vicia villosa Lectin, Wheat Germ Agglutinin, Succinylated Wheat Germ Agglutinin or any combination thereof. For example, the compound for detecting acrosomal modifications can comprise lectin peanut agglutin (PNA).

In various embodiments, the method for determining plasma membrane disruption and/or acrosomal modifications in the sample comprises comparing the plasma membrane disruption and acrosomal modifications in the sample to plasma membrane disruption and/or acrosomal modifications in (a) a non-capacitated sperm sample or (b) a post capacitated sperm sample. In various embodiments, the post capacitated sperm sample comprises a sperm sample that has undergone in vitro capacitation in a defined capacitation medium. As used herein, the term “defined capacitation medium” comprises a cell growth medium suitable for culturing and maintaining sperm cells that further comprises additives known to elicit capacitation.

Suitable additives for the defined capacitation medium may comprise, for example, sodium bicarbonate (NaHCO₃). In some embodiments, the capacitation medium comprises from about 1 mM to about 20 mM sodium bicarbonate. In some embodiments, the capacitation medium comprises a low concentration of sodium bicarbonate (i.e., about 1 mM to about 5 mM). For example, the capacitation medium may comprise about 2 mM sodium bicarbonate. In other embodiments, the capacitation medium may comprise a high concentration of sodium bicarbonate (i.e., about 10 mM to about 20 mM). For example, the capacitation medium may comprise about 15 mM sodium bicarbonate.

The capacitation medium may also comprise other additives or components necessary for cell health. These may include salts, sugars, sugar alcohols, buffers, amino acids, antibiotics, antimicrobials, proteins or any combination thereof. For example, the capacitation medium may comprise salts (in addition to sodium bicarbonate) such as NaCl, NaH₂PO₄, KCl, MgCl₂, CaCl₂. It may include buffers such as HEPES (4-(2-hydroxyethyl)-1-piperazineethanesulfonic acid), MOPS (3-(N-morpholino)propanesulfonic acid), Tris (tris(hydroxymethyl)aminomethane) or MES (2-(N-morpholino)ethanesulfonic acid) and sugars or sugar alcohols like glucose and sorbitol. It may further comprise amino acids such as Na-pyruvate, Na-lactate or proteins such as bovine serum albumin. In some aspects polyvinyl alcohol (PVA) may be used in addition to, or in lieu of, bovine serum albumin. The composition can further comprise antibiotics such as gentamycin and penicillin.

An exemplary “defined” capacitation media is provided herein in the table below.

Defined Capacitation Medium

| | Concentration | | Exemplary | |
|---|---|---|---|---|
| Reagent | Range | | Concentration | |
| NaCl | 100-130 | mM | 114 | mM |
| NaH₂PO₄ | 0.1-1 | mM | 0.34 | mM |
| KCl | 1-5 | mM | 3.2 | mM |
| MgCl₂ | 0.1-1 | mM | 0.5 | mM |
| Glucose | 5-15 | mM | 11 | mM |
| Na-pyruvate | 1-10 | mM | 5.2 | mM |
| Na-lactate | 10-30 | mM | 20 | mM |
| HEPES | 5-15 | mM | 10 | mM |
| Sorbitol | 10-20 | mM | 12 | mM |
| Gentamycin | 15-30 | mM | 21 | mM |
| Penicillin | 0.05 to 0.5 | mM | 0.174 | mM |
| CaCl₂ | 1 to 5 | mM | 2 | mM |
| NaHCO₃ | 1 to 20 | mM | 2 mM or 15 mM | |
| polyvinyl alcohol (PVA) | 0.001 to 0.5 | mM | 0.01 | mM |

| | | |
|---|---|---|
| bovine serum albumin (BSA) | 1 to 5% | 2% |

In some embodiments, the defined capacitation medium comprises heparin, caffeine, calcium ionophore or any combination thereof.

In various embodiments, high fertility sperm and/or sperm associated with zinc signatures 1 and/or 2 are characterized by reduced plasma membrane disruption and reduced acrosomal modifications. In various embodiments, low fertility sperm and/or sperm associated with zinc signatures 3 and/or 4 are characterized by increased plasma membrane disruption and increased acrosomal modifications. In various instances, the compounds used to identify disrupted plasma membranes and compromised/modified acrosomes show increased labeling in the presence of disrupted plasma membranes and compromised/modified acrosomes, respectively.

The correlation between sperm capacitation and fertility is one of timing. A fertile spermatozoa must be fully capable of undergoing capacitation, but not do so when in storage or during transportation. Therefore, in various embodiments, a sperm source is identified as fertile or infertile depending on whether sperm obtained from it are capable of undergoing capacitation. To determine this, in various embodiments, a sperm sample obtained from the source is exposed to in vitro capacitation conditions (such as, for example, the defined capacitation medium described above) and labeled with a zinc probe. In various embodiments, the sperm source is identified as a fertile sperm source when it achieves an increased subpopulation of spermatozoa having zinc signature 3 and/or zinc signature 4 after in vitro capacitation, as compared to a sample from the sperm source not put through in vitro capacitation. In certain such embodiments, the sample exposed to in vitro capacitation is from a sperm source from which previously tested samples have exhibited zinc signature 1 or signature 2. One of the previously tested samples, or a new sample from the same source, can be exposed to in vitro capacitation. When sperm from such source exhibits zinc signature 3 or 4 after in vitro capacitation, it demonstrates that sperm from the sperm source have the potential to transition to signature 3 or 4, which indicates the sperm source is fertile.

In various embodiments, the sperm source may be a mammalian sperm source or an in vitro sperm source. The mammalian sperm source can an animal or a reservoir of collected semen to be used for artificial fertilization and/or in vitro fertilization. An in vitro sperm source can comprise spermatozoa that underwent spermatogenesis in vitro (i.e., is not simply collected from a live animal). As described above, the sperm source may be identified as fertile or infertile based on the fertility of the sperm sample obtained from it. In various embodiments, the methods described herein may further comprise selecting a fertile source of spermatozoa for further use in artificial insemination, in vitro fertilization and/or breeding. Likewise, the methods described herein my further comprise using the fertile source of sperm in artificial insemination, in vitro fertilization and/or breeding.

In some embodiments, the identified fertile sperm source is a mammalian source and in various embodiments, the methods may further comprise using the mammalian source for any purpose in which a fertile mammal is useful.

### Image-Based Flow Cytometry

In various embodiments, the zinc presence and/or localization may be analyzed visually (i.e., using microscopy), optionally in connection with computer analysis (e.g. computer-assisted sperm analysis systems). In certain embodiments, the zinc signatures are analyzed using flow cytometry. Preferably, the flow cytometry comprises image-based flow cytometry, which combines features of flow cytometry and fluorescent microscopy to allow for rapid analysis of multiple fluorescent signals from a large group of cells. Traditional flow cytometry measuring whole cell FZ3 (i.e., zinc) intensity cannot distinguish between some zinc signatures (particularly Signatures 1 and 2). This is because the strong zinc signal from the sperm head can mask variations in smaller cellular regions, such as the sperm tail.

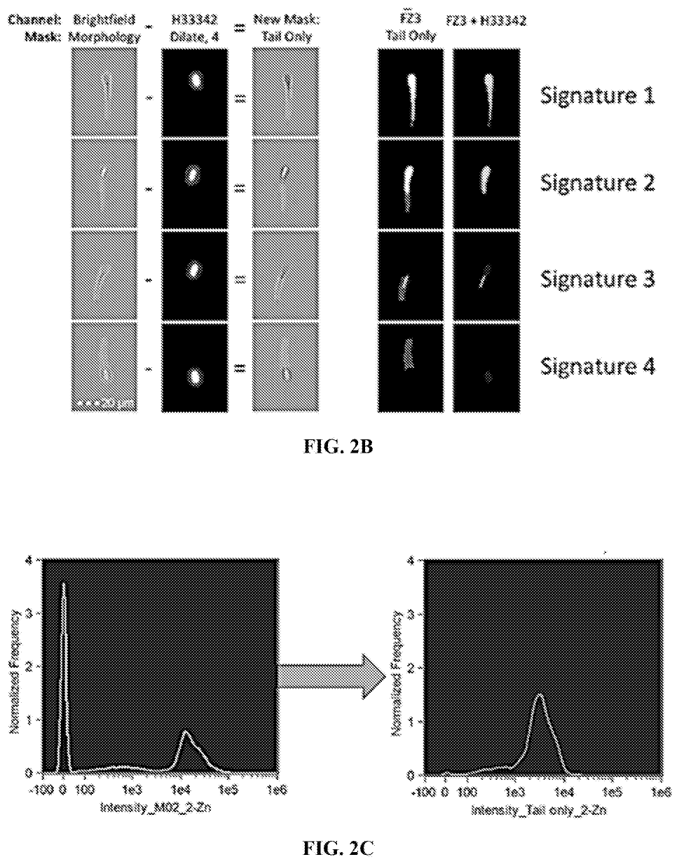

In the image-based flow cytometry methods described herein and depicted in representative FIGS. 2A-2D, masking, gating and other corrections are employed to isolate relevant zinc signals that enable robust and accurate classification of fertile and infertile sperm.

In various embodiments, using the flow cytometry comprises correcting for cytoplasmic droplets containing zinc on the spermatozoa in the sample. In various embodiments, the flow cytometry can comprise gating and/or masking of the flow cytometry data. The gating, for example, can comprise single cell gating. The masking, for example, can comprise using a mask that discards laterally aligned spermatozoa and, optionally, only analyzes the sperm tail. In some embodiments, the mask only analyzes the sperm tail.

In various embodiments, the methods described herein further comprise labeling the sample with a DNA dye prior to the identifying step. In these embodiments, the mask may be created by subtracting fluorescence of the DNA dye from a mask that results in sperm tail identification. In some instances the sperm nucleus, which is identified by DNA dye, does not encompass the entire sperm head. Therefore, in certain embodiments, a dilation of the DNA dye mask (i.e., by increasing the mask boundary by a certain amount, such as 4 pixels) is subtracted from the mask that results in sperm tail identification. This method allows for removal of the high zinc signal localized in the sperm head, allowing for more accurate analysis of the zinc signal in the sperm tail.

In various embodiments, the mask that results in sperm tail identification comprises a brightfield image, a side scatter image or any other mask that encompasses the entire spermatozoon.

In various embodiments, the DNA dye comprises a DNA dye that can permeate living cells (e.g., cells with intact plasma membranes). In some embodiments, the DNA dye can comprise a Hoeschst dye. For example, the DNA dye can comprise Hoescht 33342, Hoescht 33258, or Hoescht 34580. In some embodiments, the DNA dye comprises Hoescht 33342.

### II. Methods for Improving Sperm Fertility

Also provided are methods of improving sperm or semen fertility. In various embodiments, the methods comprise adding exogenous zinc ions to a sample of sperm or semen. In various embodiments, a sperm is identified as infertile, subfertile, or fertile according to any methods described herein. In certain embodiments, these methods of improving sperm or semen fertility comprise preventing or inhibiting premature capacitation while the sperm or semen is transported or stored.

### III. Sperm Extender Compositions and Use Thereof

Provided herein are compositions for inhibiting capacitation in a sperm sample. The compositions comprise at least about 0.5 M to about 5 M of exogenous zinc ions. For example, the composition may comprise about 1 M to about 3 M of exogenous zinc ions. In various embodiments, the exogenous zinc ions can be provided in the form of a salt (i.e., zinc chloride, ZnCl₂). Other forms of zinc include ZnCO₃, Zn₃(PO₄)₂, zinc acetate, zinc citrate, and ZnSO₄.

The compositions herein may further comprise a carrier or an excipient (i.e., a pharmaceutically acceptable carrier). Pharmaceutically acceptable excipients are identified, for example, in The Handbook of Pharmaceutical Excipients, (American Pharmaceutical Association, Washington, D.C., and The Pharmaceutical Society of Great Britain, London, England, 1968). Additional excipients can be included in the pharmaceutical compositions of the invention for a variety of purposes. These excipients can impart properties which enhance retention of the compound at the site of administration, protect the stability of the composition, control the pH, facilitate processing of the compound into pharmaceutical compositions, and so on. For example, the composition may comprise a sperm encapsulation component such as an unbranched polysaccharide extract (e.g., poly-1-lysine or an alginate formed by the condensation of guluronic and mannuronic acid). Other excipients include, for example, cryoprotectants (e.g., glycerol or dimethylacetamide), fillers or diluents, surface active, wetting or emulsifying agents, preservatives, agents for adjusting pH or buffering agents, thickeners, colorants, dyes, flow aids, non-volatile silicones, adhesives, bulking agents, flavorings, sweeteners, adsorbents, binders, disintegrating agents, lubricants, coating agents, and antioxidants.

In various embodiments, a sperm sample comprising a zinc composition described herein is provided. In other embodiments, a semen medium or extender is provided comprising any zinc composition described herein. In some embodiments, zinc may be added to any commercially available semen extender. The commercially available semen extender may comprise a SPERMVITAL product from Sperm Vital. Preferably, the semen medium or extender has the capability of preventing premature capacitation of spermatozoa in the sample.

Therefore, in various embodiments a method is provided for preserving semen for in vitro fertilization, artificial insemination, cryopreservation, or sexed spermatozoa/semen usage, the method comprising adding the zinc composition described herein to the semen and/or sperm media to prevent spontaneous sperm capacitation.

Having described the invention in detail, it will be apparent that modifications and variations are possible without departing from the scope of the invention defined in the appended claims.

### EXAMPLES

The following non-limiting examples are provided to further illustrate the present invention.

### Materials and Methods

The following materials and methods were used in Examples 1-4 below.

### Reagents

All reagents unless otherwise noted were from Sigma. FluoZin™-3, AM (FZ3; zinc probe) from ThermoFisher (F24195) was reconstituted with DMSO to a stock solution of 500 μM. Lectin PNA (Arachis hypogea/peanut agglutinin) conjugated to Alexa Fluor™ 647 (PNA-AF647) from Invitrogen™ (L32460). Fluo-4 NW (calcium probe) from ThermoFisher (F36206) was reconstituted using kit provided assay buffer. Hoechst 33342 (H33342) from Calbiochem (382065) was reconstituted with H₂O to a stock solution of 18 mM. Propidium Iodide (PI) from Acros Organics (AC440300010) was reconstituted with H₂O to a stock solution of 1 mg mL⁻¹. Proteasomal inhibitors were from Enzo Life Sciences: MG132 (BML-PI102) was reconstituted with DMSO to a stock solution of 20 mM; Epoxomicin (Epox, BML-PI127) was reconstituted to a stock solution of 20 mM (using MG132 stock); and clasto-Lactacystin β-Lactone (CLBL, BML-PI108) was reconstituted with DMSO to a stock solution of 5 mM. Zn-chelator TPEN from Tocris (16858-02-9) was resuspended with 1:100 EtOH:H₂O to a stock solution of 1 mM. Bovine serum albumin (BSA) was from Sigma (A4503). Anti-phosphotyrosine antibody, clone 4G10® was from EMD Millipore (05-321).

### Semen Collection and Processing

Boar semen collection was performed under the guidance of approved Animal Care and Use (ACUC) protocols of the University of Missouri-Columbia. Boar semen for the fertility trial was collected, extended, and shipped by overnight parcel from a private boar stud following their established standard operating procedures and was not blinded. Boar collection was performed using standard two gloved hand technique⁵². Only ejaculates with greater than 80% motility were used and no randomization was necessary as only one boar was studied at a time. The sperm rich fraction of boar ejaculate was used, except all IVC proteasomal inhibition studies used the pre-sperm rich fraction, which had increased viability and sensitivity to 26S inhibition. Semen was immediately extended, within 2° C., five times in Beltsville thawing solution (BTS) semen extender. Sperm concentration was then determined using a hemocytometer. All washes were performed with a swing hinge rotor centrifuge at 110×g for 5 minutes. Number of washes and g-force used were minimized as these were found to compromise results. Frozen-thawed bull spermatozoa were processed similarly as boar spermatozoa after being thawed for 45 seconds in 35° C. water bath. For human spermatozoa, sperm donors signed informed consent and the samples were coded as to make the donors unidentifiable to researchers. All human sperm samples were handled and processed strictly as stipulated by an approved Internal Review Board (MU IRB) protocol. Donors were recruited by placing an advertisement for new fathers in the university mass e-mail newsletter. All semen was collected onsite at the Missouri Center for Reproductive Medicine and Fertility clinic. Samples were then transported to the laboratory for analysis.

### In Vitro Capacitation

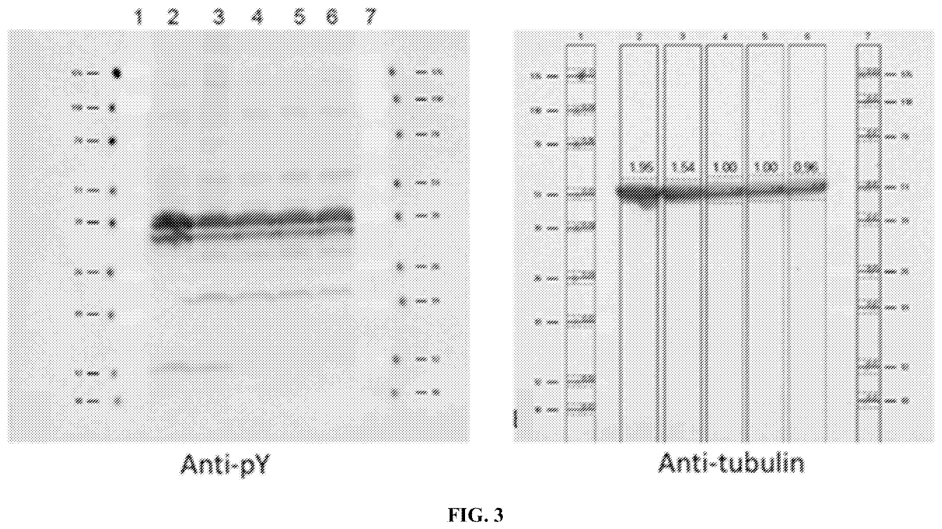

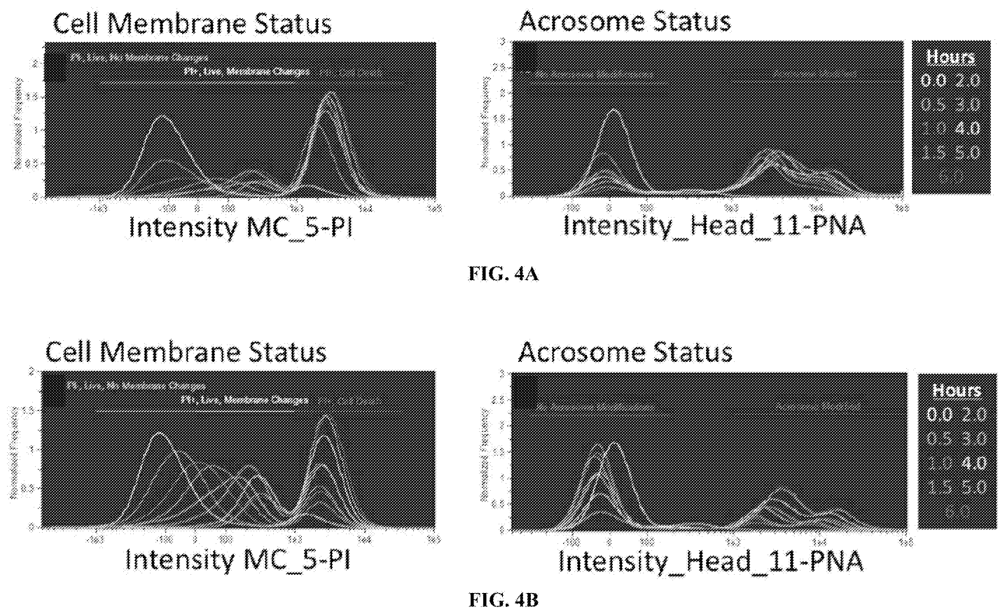

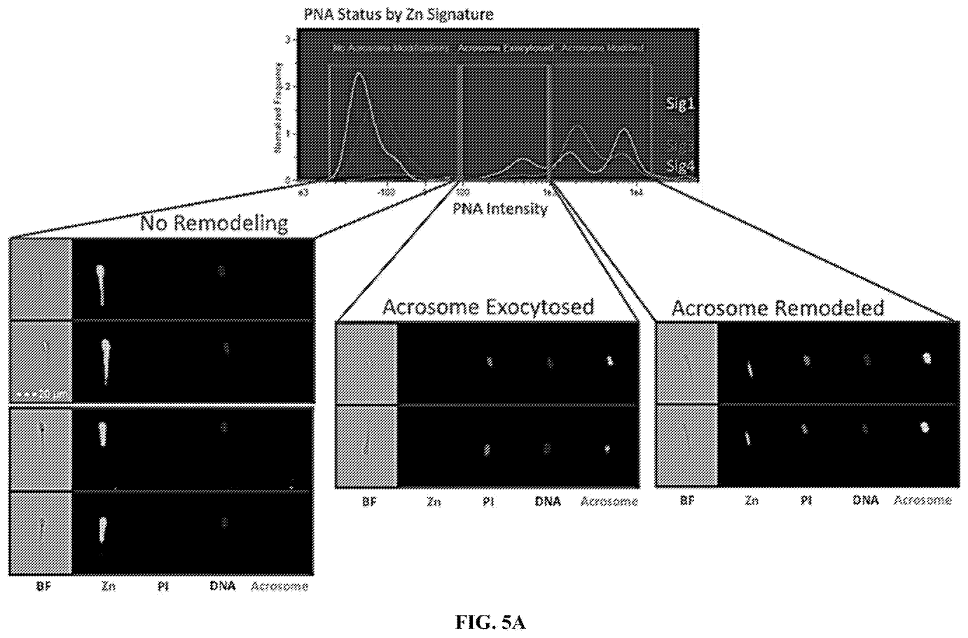

Fresh boar spermatozoa were capacitated using a protocol that rendered them capable of recognizing and binding to ZP, as well as undergoing acrosomal exocytosis and penetrating the oocyte ZP¹¹. IVC-induced protein tyrosine phosphorylation changes are shown in FIG. 3; acrosomal status and plasma membrane changes are shown in FIG. 4A and FIG. 4B. Briefly, spermatozoa were washed of seminal plasma once with noncapacitating media (NCM), a modified TL-HEPES medium, free of calcium dichloride (CaCl₂) and addition of 11 mM D-glucose, with pH adjusted to 7.2. Spermatozoa were then resuspended in 0.5 mL in vitro capacitation (IVC) media, TL-HEPES-PVA supplemented with 5 mM sodium pyruvate, 11 mM D-glucose, 2 mM CaCl₂, 2 mM sodium bicarbonate, and 2% (m/v) bovine serum albumin, and incubated in a 37° C. water bath for 4 hours, with sperm rotation performed every 60 minutes. Control incubations under non-IVC conditions used NCM. Proteasome inhibitors (100 μM MG-132 and 10 μM Epox/CLBL/MG-132) were mixed with IVC media prior to sperm pellet resuspension. 100 μM M MG-132 and ‘100 μM’ vehicle contained 0.5% (v/v) DMSO. 10 μM Epox/CLBL/MG-132 and ‘10 μM’ vehicle contained 0.3% (v/v) DMSO. PVA (polyvinyl alcohol) helped to reduce sperm aggregation and spermatozoa were pipetted repeatedly to dissociate sperm aggregates in a satisfactory manner prior to IBFC data acquisition. To confirm normal capacitation in the experimental IVC media, compared to 15 mM sodium bicarbonate IVC media, normal hyperactivation status was recorded and tyrosine phosphorylation status was normal (FIG. 3). Unlike murine or rodent sperm tyrosine phosphorylation, porcine tyrosine phosphorylation is much more modest, with less prominent changes during the course of capacitation. Therefore new bands after capacitation appear only at the molar weights of 32 kDa (acrosin binding protein), and 21 kDa protein (phospholipid hydroperoxide glutathione peroxidase). These results are in accordance with previous studies⁵³⁻⁵⁵. Final acrosome and plasma membrane modification status is similar at end of IVC regardless of the two IVC treatment conditions; however, the rate of change and cell death is faster in 15 mM sodium bicarbonate containing medium than experimental IVC medium (FIGS. 4A and 4B). Altogether, this supports the use of experimental IVC medium over 15 mM sodium bicarbonate containing medium to display the prolonged lifespan of spermatozoa as seen in in vivo capacitation.

### TPEN Zn Chelation

Zn²⁺ chelation was performed using TPEN (membrane permeable). 10 μM TPEN was incubated with 40 million sperm per mL for 1 hour. Stock TPEN: 1 mM in 1:100 EtOH:H₂O.

### Multiplex Fluorescence Probing

Upon 4 hours of IVC, sample size of 100 μL (4 million spermatozoa) were incubated 30 minutes with 1:200 H33342, 1:200 PI, and 1:100 FZ3 for epifluorescence microscopy. Lower probe concentrations were necessary for IBFC due to camera detection differences, thus 1:1000, 1:1000, and 1:500 were used, respectively, with inclusion of 1:1000 PNA-AF647. For Fluo-4 calcium probe, we followed manufacturer protocol using identical cell concentrations. Spermatozoa were then washed of probes once and resuspended in corresponding IVC treatment media to allow complete de-esterfication of intracellular AM esters, as suggested by ThermoFisher's FZ3 protocol, followed by an additional wash and resuspended in 100 μL PBS for IBFC analysis (or added to a slide for epifluorescence microscopy imaging).

### Epifluorescence Microscopy Imaging

Live spermatozoa were imaged using a Nikon Eclipse 800 microscope (Nikon Instruments Inc.) with Cool Snap camera (Roper Scientific, Tucson, Ariz., USA) and MetaMorph software (Universal Imaging Corp., Downington, Pa., USA). Images were adjusted for contrast and brightness in Adobe Photoshop CS5 (Adobe Systems, Mountain View, Calif.) to match the fluorescence intensities viewed through the microscope eyepieces.

### Image-Based Flow Cytometric Data Acquisition

IBFC data acquisition was performed following previous methodology⁵⁶. Specifically, using a FlowSight flow cytometer (FS) fitted with a 20× microscope objective (numerical aperture of 0.9) with an imaging rate up to 2000 events per sec. The sheath fluid was PBS (without Ca²⁺ or Mg²⁺). The flow-core diameter and speed was 10 μm and 66 mm per sec, respectively. Raw image data were acquired using INSPIRE® software. To produce the highest resolution, the camera setting was at 1.0 μm per pixel of the charged-coupled device. In INSPIRE® FS data acquisition software, two brightfield channels were collected (channels 1 & 9), one FZ3 image (channel 2), one PI image (channel 5), one side scatter (SSC; channel 6), one H33342 (channel 7), and one PNA-AF647 image (channel 11), with a minimum of 10,000 spermatozoa collected. The following lasers and power settings were used: 405 nm (to excite H33342): 10 mW; 488 nm (to excite FZ3): 60 mW; 561 nm (to excite PI): 40 mW, 642 nm (to excite PNA-AF647): 25 mW; and 785 nM SSC laser: 10 mW.

### IBFC Data Analysis

Data were analyzed using IDEAS® analysis software from AMNIS EMD Millipore. Gating approach used standard focus and single cell gating calculations created by IDEAS software (FIG. 2A, left and middle panels). Briefly, initial events collected from FlowSight data acquisition were gated for cells in focus as a function of brightfield gradient RMS (a calculation of image crispness) (FIG. 2A, left panel). Events in focus were further gated to analyze only a single spermatozoa by plotting H33342 fluorescence area by aspect ratio (FIG. 2A, middle panel). To further clean up data for analysis, Feature Finder function was used to discover image-based calculations to discard spermatozoa laterally aligned with the camera, as opposed to anteriorly/posteriorly aligned. Specifically, to differentiate and gate out laterally aligned spermatozoa, a plot of brightfield standard deviation by brightfield H entropy was used (FIG. 2A, right panel). Traditional flow cytometric analysis methods does not allow to distinguish Signature 1 and 2 based on whole cell FZ3 intensity, therefore creating a mask that only analyzes the sperm tail proved to be key in distinguishing these two populations. Such mask was created by taking a morphology mask of the brightfield (FIG. 2B, first column), subtracting a 4-pixel dilation of H33342 (FIG. 2B, second column), resulting in a mask to analyze fluorescence in the tail region only (FIG. 2B, third and fourth columns). Mask dilation of H33342 fluorescence was necessary because H33342 labeling of the sperm nucleus did not cover the entirety of the sperm head, where FZ3 signal was high. This combined gating and masking strategy shown in FIG. 2C provided robust clean data, ready for signature analysis by plotting FZ3 intensity of this masked region, which is impossible with traditional flow cytometry. Gating boundaries for signatures 1, 2, and 3 were determined by the population segregations of fresh/ejaculate and IVC+vehicle treatments with signature status confirmed in the image gallery. Gating boundaries between signatures 3 and 4 were less evident in histograms and placed where spermatozoa lost FZ3 signal as determined using the image gallery.

### Sequential Sperm Extraction Treatment

Approximately 200 million washed spermatozoa were used per single treatment, which was conducted by adding 100 μL of a relevant reagent; proteinase and phosphatase inhibitors included. In the first step, PBS was added to the sperm pellet, allowed to incubate on ice for 30 minutes and spun. In second step, the pellet was reused and 0.75 M KCl in PBS was added and incubated on ice for 30 minutes and spun down. Pellet was washed once with PBS to remove residual salt, and reused in the third step for treatment with 30 mM n-octyl-β-D-glucopyranoside (OBG) in PBS. The sperm after each treatment step were analyzed for their zinc signature.

### Western Blotting (WB)

Sperm pellets (15 million spermatozoa per pellet) were mixed with reducing SDS-PAGE loading buffer, boiled for 5 min and briefly spun at 5000 g. The SDS-PAGE was carried out on a 4-20% gradient gels (PAGEr Precast gels; Lonza Rockland, Rockland, Me., USA) as previously described.¹³ The molecular masses of the separated proteins were estimated by using prestained Prosieve protein colored markers (Lonza Rockland) run in parallel. After SDS-PAGE, proteins were electro-transferred onto a PVDF Immobilon Transfer Membrane (Millipore, Bedford, Mass., USA) using an Owl wet transfer system (Fisher Scientific) at a constant 50 V for 4 h for immunodetection.¹³

### Statistics

All results are presented as mean±standard error. SAS 9.4 GLM procedure and Duncan's Multiple Range test was used to analyze the replicates. Bartlett and Leven tests found the sample set to be homogenous.

### Animal Models

Domestic (Sus scrofa) boars 2 years of age were used for all experiments performed. Domestic (Bos taurus) bulls 3 years of age were used to confirm zinc signature presence.

### Example 1

### Mammalian Spermatozoa Possess Four Distinct Zinc Signatures

Image-based flow cytometry (IBFC) and epifluorescence microscopy were used to trace the sperm zinc signature using Zn-probe Fluo Zin™-3 AM (FZ3), DNA stain Hoechst 33342, acrosomal remodeling detecting lectin PNA (Arachis hypogea/peanut agglutinin) conjugated to Alexa Fluor™ 647 (PNA-AF647), and live/dead cell, plasma membrane-integrity reflecting DNA stain propidium iodide (PI), which is taken up by exclusively by cells with a compromised/remodeled plasma membrane. The IBFC, which combines the fluorometric capabilities of conventional flow cytometry with high speed-multi-channel image acquisition, proved to be advantageous due to the high presence of Zn²⁺ in sperm cytoplasmic droplets and seminal debris, which otherwise would distort traditional flow cytometry results. A unique gating and masking strategy was developed to ensure unbiased data analysis (FIG. 2A-2D). Analyses were performed using the initial, pre-sperm rich fraction of ejaculates, which had highest sperm viability/plasma membrane integrity, repeatability and sensitivity to proteasomal inhibition compared to secondary, sperm-rich fraction which appeared more prone to spontaneous capacitation and loss of plasma membrane integrity

Four distinct types of sperm zinc signatures were found conserved across boar (FIG. 1A), bull (FIG. 1B), and human spermatozoa (FIG. 1C): high Zn′ presence in the sperm head and whole sperm tail (signature 1, non-capacitated spermatozoa, upper left panels in FIGS. 1A, 1B and 1C), medium-level (based on relative intensity of fluorescence in FlowSight measurements) Zn²⁺ presence in both the sperm head and sperm tail midpiece (signature 2, spermatozoa undergoing capacitation; upper right panels in FIGS. 1A, 1B, and 1C), Zn²⁺ presence in the midpiece only (signature 3, capacitated state signature in spermatozoa that underwent capacitation and may be dying; bottom left panels in FIGS. 1A, 1B and 1C), and no Zn²⁺ presence (signature 4, spermatozoa with compromised/remodeled plasma membrane; bottom right panels in FIGS. 1A, 1B and 1C). Spermatozoa after 72 hours of storage in Beltsville Thaw Solution (BTS semen extender) show varied zinc signatures (FIG. 1D)

### Example 2

### Zinc Signature is Indicative of Capacitation Status In Vitro

A drawback to commonly used 15 mM sodium bicarbonate in vitro capacitation media is rapid sperm death (as compared to in vivo sequential capacitation¹⁰), illustrated in the time course study by a shift to PI+ cell death flow cytometry gating (FIG. 4A, left panel) and rapid acrosomal modification (FIG. 4A, right panel). In the interest of emulating in vivo sperm life span and sequential capacitation as a fertility diagnostic method, a previously described capacitation medium¹¹ was used. This medium had low (2 mM) sodium bicarbonate and increased sodium pyruvate (5 mM) and prolonged sperm viability (FIG. 4B, left panel) and elicited similar hyperactivation while achieving hallmark acrosomal modification (FIG. 4B, right panel).

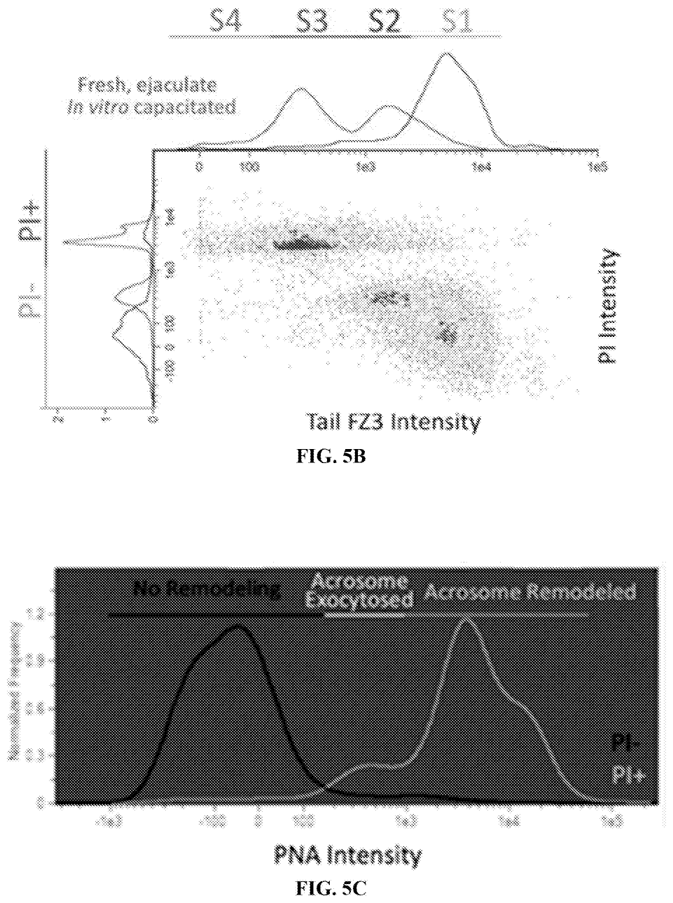

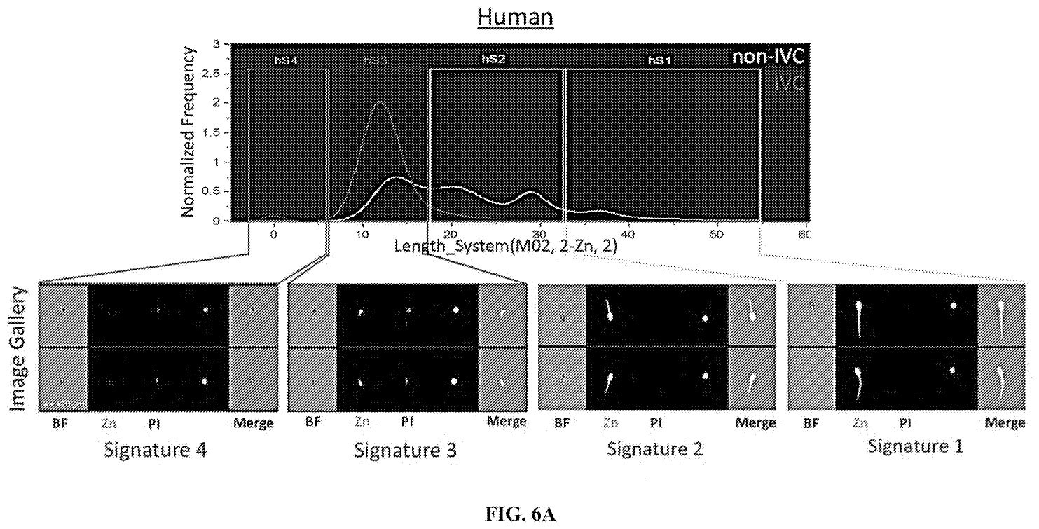

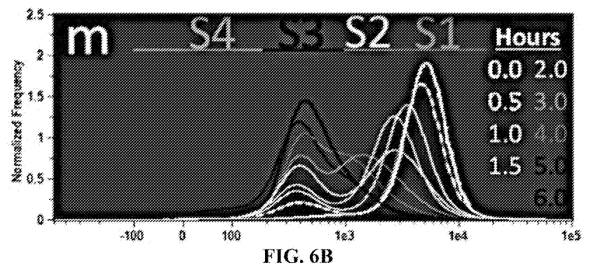

Most spermatozoa in zinc signature 1 and 2 states had no capacitation-like acrosomal remodeling (93.0%±6.8% and 95.0%±2.6%, data presented as mean±s.d.; 10,000 cells analyzed per treatment, n=3 biological replicates) compared to zinc signature 3 and 4 (11.1%±5.8% and 7.0%±9.9%; P<0.0001, as determined by the General Linear Model (GLM) procedure). Capacitation-like acrosomal remodeling was most prevalent with zinc signatures 3 and 4 (81.0%±8.5% and 62.2%±12.9%) compared to zinc signatures 1 and 2 (4.0%±4.7% and 3.4%±2.9%; P<0.0001, as determined by the GLM procedure, 4 biological replicates; 10,000 spermatozoa analyzed per treatment). Acrosome exocytosis occurred within the subpopulation of spermatozoa with zinc signature 4 (30.7%±3.0%) and was greater than zinc signatures 1, 2, and 3 (3.0%±2.6%, 1.6%±1.2%, 7.9%±2.9%; P<0.001, as determined by the GLM procedure; FIG. 5A). These results are summarized in Table 1 below. The data is presented as mean±s.d. (3 biological replicates). Values with different uppercase superscripts (^{A,B,C,D}) indicate significant difference of the acrosomal status (P-value≤0.0001) and lowercase superscripts (^{a,b,c}) indicate significant difference of zinc signatures (P-value≤0.0002) as determined by the GLM procedure in SAS 9.4. Both PI+ and PI− cells were included in this analysis. A total of 10,000 cells were measured for each replicate.

TABLE 1
Statistical analysis of zinc signature and acrosomal status

| | No Remodeling | Remodeled | Exocytosed |
|---|---|---|---|
| Signature 1 | 93.0% ± 6.8%^{Aa} | 4.0% ± 4.7%^{Ba} | 3.0% ± 2.6%^{Ba} |
| Signature 2 | 95.0% ± 2.6%^{Aa} | 3.4% ± 2.9%^{Ba} | 1.6% ± 1.2%^{Ca} |
| Signature 3 | 11.1% ± 5.8%^{Ab} | 81.0% ± 8.5%^{Bb} | 7.9% ± 2.9%^{Aa} |
| Signature 4 | 7.0% ± 9.9%^{Ab} | 62.2% ± 12.9%^{Bb} | 30.7% ± 3.0%^{Cb} |

As sperm plasma membrane-integrity decreased, signaled by increased propidium iodide (PI) labeling, the zinc patterns progressed to signatures 3 and 4 (FIG. 5B and FIG. 5C). The superimposition of zinc ion labeling and PI labeling in flow cytometric scatter plots allows for the subdivision of spermatozoa within the boar ejaculate into four subpopulations (FIG. 5B). Cell membrane changes heralded by PI incorporation in the sperm head at capacitation are concomitant with acrosomal remodeling signaled by lectin PNA binding, even though they occur at the opposite poles of the sperm head (FIG. 5C). Further, sperm showed the capability of transitioning between zinc signatures during capacitation. A strong shift from signatures 1 and 2 to signature 3 is apparent for IVC treated spermatozoa. FIG. 6A shows human sperm zinc signatures associated with non-capacitated and in vitro capacitated sperm; IVC treated spermatozoa have a larger percentage of signature 3 than non-capacitated samples. This shift occurs rapidly as seen in FIG. 6B which shows a time course of zinc signatures of sperm at various time points during in vitro capacitation. There is a clear shift from signature 1 and 2 to signatures 3 and 4 during the time course. Hyperactivated spermatozoa, capable of recognizing and binding the oocyte zona pellucida have zinc signature 2, in which the transition from Signature 1 to 2 occurs within the first 30-60 minutes of IVC (FIG. 6B). These results suggest that disproportional representation of signatures 3 and 4, associated with sperm capacitated state and death, in non-capacitated sperm samples may indicate low fertility ejaculates, but that an increased representation of these signatures 3 and 4 in a capacitated sample (relative to levels prior to capacitation) may indicate high fertility associated with the ability to undergo capacitation.

### Example 3

### 26S Proteasome Modulates Zinc Signature Capacitation Shift

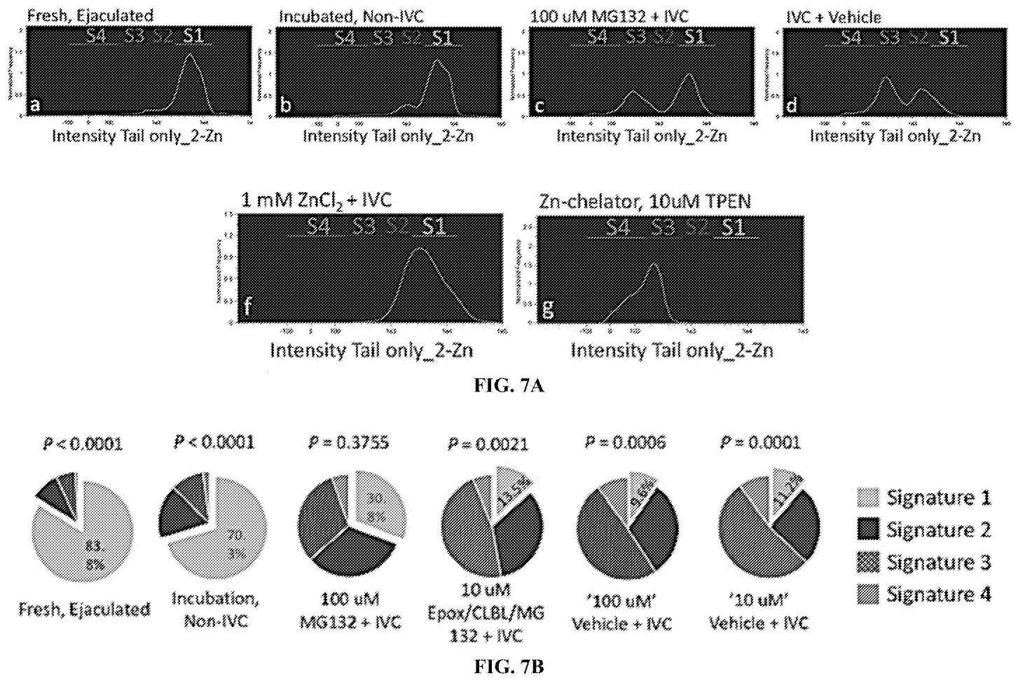

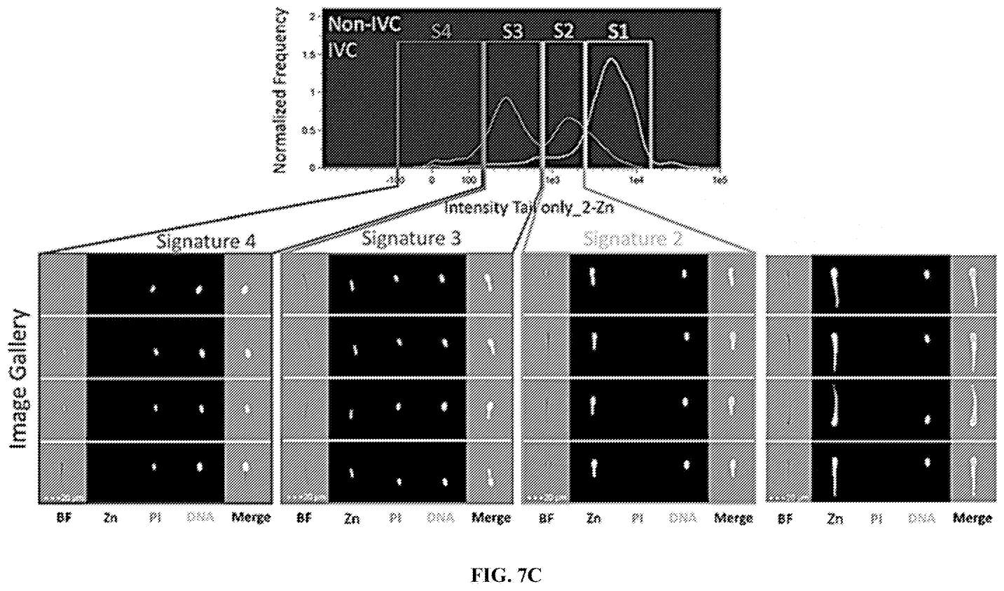

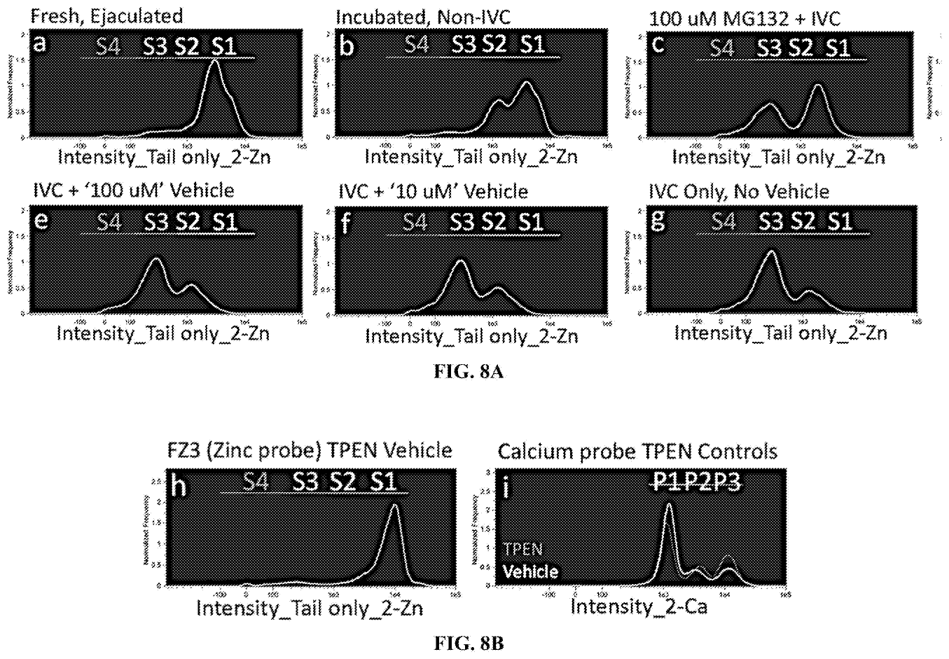

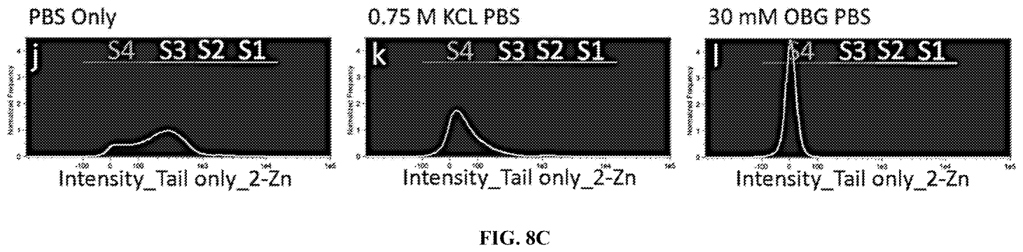

FIG. 7A shows representative zinc signature histograms as determined from IBFC analysis of sperm obtained or stored in various conditions (as indicated). FIG. 8A shows representative zinc signature histograms determined the same way from an independent second biological replicate. Fresh, ejaculated boar spermatozoa mostly had signature 1, (83.8%±3.1%; data presented as mean±s.e.m.; 10,000 cells analyzed per treatment, n=3 biological replicates; FIG. 7A). A small portion of spermatozoa incubated in non-IVC media for 4 hours at 37° C. progressed to signature 2 (FIG. 7A) as compared to spermatozoa in the same media incubated at room temperature to emulate the conditions of artificial insemination (FIG. 7A), suggesting that some spermatozoa undergo temperature-induced, early stage capacitation. When proteasome inhibitor MG-132 was added to IVC conditions to reduce sperm proteasome activity as previously described^{12,13}, a significantly higher portion of spermatozoa retained signature 1 when using the pre-sperm rich fraction (FIG. 7A) as compared to IVC+vehicle (P=0.0271; when signatures 1 & 2 combined P=0.0008, as determined by Duncan's Multiple Range test). After 4 hours of IVC, the zinc signature changed to mostly signature 3 (49.4%±7.9%), with a small portion of spermatozoa having signature 2 (31.3%±12.3%; FIG. 7A). Remarkably, manipulation of sperm Zn²⁺ content during IVC reset the zinc signature (FIG. 7A). Spermatozoa retained signature 1 with addition of 1 mM ZnCl₂ to IVC medium (FIG. 7A). Cell-permeant Zn²⁺ chelator N,N,N′,N′-Tetrakis(2-pyridinylmethyl)-1,2-ethanediamine (TPEN) removed a majority of FZ3 fluorescence to signature 3 and 4 states (FIG. 7A) compared to TPEN vehicle but TPEN did not reduce Fluo-4 calcium probe intensity compared to vehicle (FIG. 8B). Remaining FZ3 fluorescence is likely due to Zn presence, but ions tightly bound within the mitochondrial sheath, as zinc has been previously detected there by electron microscopy¹⁴. With the exception of the midpiece, zinc ions appeared to be associated with the sperm surface, as the stepwise extraction removed Zn′ tracer fluorescence early in treatment procedure (FIG. 8C).

The results for the effect of proteosomal inhibition on zinc signatures as described above (see FIG. 7A) are summarized graphically in FIG. 7B and in Table 2 below. In the table, data are presented as mean±s.e.m. (3 biological replicates). Values with different uppercase superscripts (^{A,B,C,D}) indicate significant difference between the control (fresh, ejaculated spermatozoa and vehicle controls) and treatment groups and lowercase superscripts (^{a,b,c}) indicate significant difference between signatures as determined by Duncan's Multiple Range test (P-value by treatment and signature in table). Both PI+ and PI− cells were included in this analysis. Treatment column refers to proteasomal inhibitors MG132, Clasto-Lactacystin β-Lactone (CLBL) and epoxomicin (Epox.). A total of 10,000 cells were measured for each data point.

TABLE 2
Effect of proteasomal inhibition on zinc signature

| Treatment | Signature 1 | Signature 2 | Signature 3 | Signature 4 | P-value |
|---|---|---|---|---|---|
| Fresh, | 83.8% ± 1.8%^{Aa} | 9.2% ± 0.9%^{b} | 6.1% ± 1.2%^{Ab} | 0.9% ± 0.4%^{Ac} | P < 0.0001 |
| Ejaculated | | | | | |
| Incubation, | 70.3% ± 2.5%^{Aa} | 17.3% ± 3.0%^{b} | 10.4% ± 2.7%^{Ab} | 2.0% ± 2.7%^{Ac} | P < 0.0001 |
| Non-IVC | | | | | |
| 100 μM | 30.8% ± 13.1%^{B} | 32.9% ± 19.5% | 31.0% ± 4.9%^{B} | 5.4% ± 4.9%^{AB} | P = 0.3755 |
| MG132 + IVC | | | | | |
| 10 μM Epox, | 13.5% ± 4.0%^{BC} | 33.7% ± 8.8% | 46.8% ± 3.6%^{C} | 6.1% ± 3.6%^{B} | P = 0.0021 |
| CLBL, MG132 + | | | | | |
| IVC | | | | | |
| ‘100 μM’ | 9.6% ± 3.3%^{Ca} | 31.3% ± 7.0%^{b} | 49.4% ± 4.5%^{Cc} | 9.8% ± 4.5%^{Ba} | P = 0.0006 |
| Vehicle + IVC | | | | | |
| ‘10 μM’ | 11.2% ± 2.5%^{Ca} | 25.8% ± 3.6%^{b} | 53.1% ± 3.8%^{Cc} | 9.9% ± 3.8%^{Ba} | P = 0.0001 |
| Vehicle + IVC | | | | | |
| P-value | P < 0.0001 | P = 0.4119 | P < 0.0001 | P < 0.0003 | |

### Example 4

### Zinc Signature Associated with Varied Fertility in AI Boars

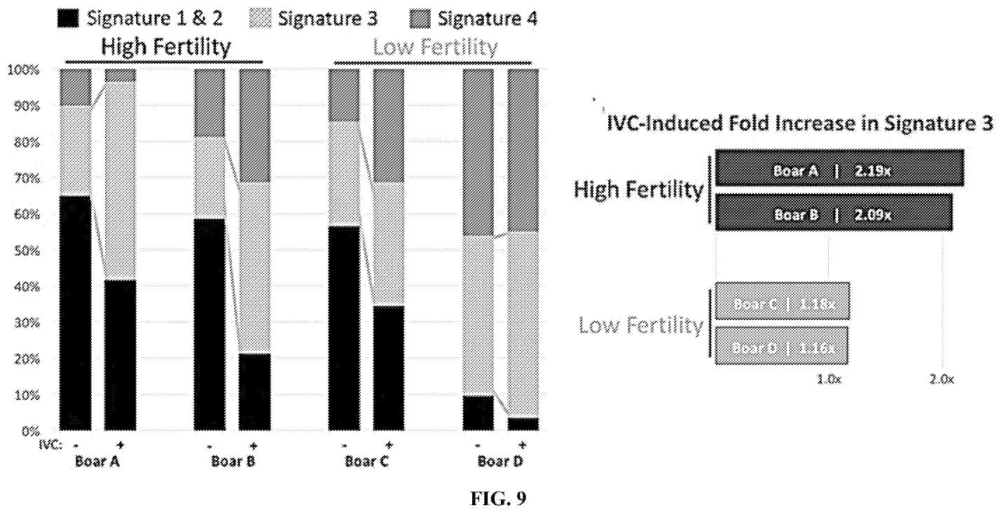

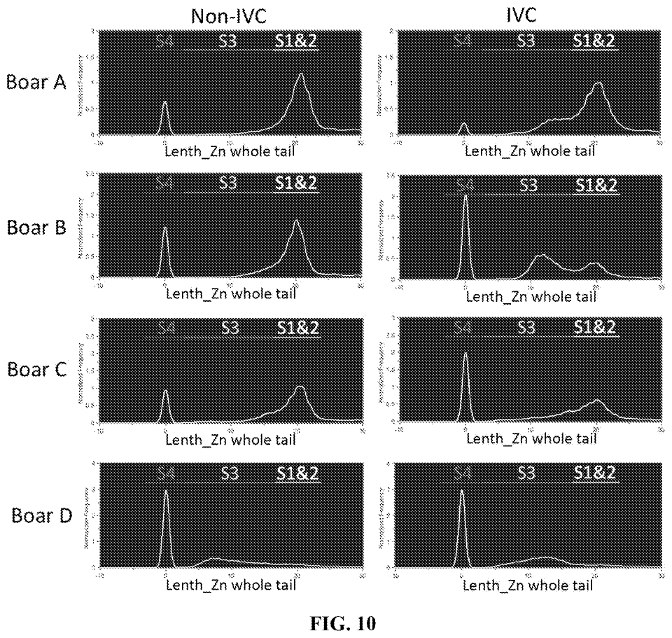

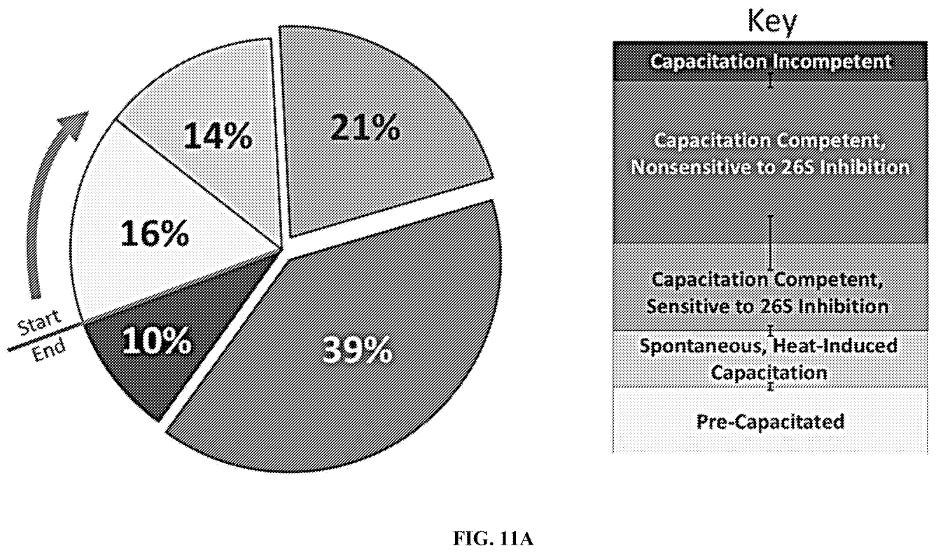

In this example, possible individual variability in sperm zinc signature in AI boars with acceptable but varied fertility was examined. In a small preliminary fertility trial (n=4 boars with known fertility in AI service; fertility records in Table 3 below), zinc signatures differed between high and low fertile boars both after IVC (FIG. 9; original histograms located in FIG. 10). Boars with high fertility have double the amount of signature 3 spermatozoa prevail after IVC (as percentage of population) as opposed to minimal signature 3 increase in low fertility boars (FIG. 9)

TABLE 3
Boar fertility trial records.

| Boar | Farrowing Rate | Average Litter Size | Services (n) |
|---|---|---|---|
| Boar A | 90.5% | 10.03 | 42 |
| Boar B | 89.5% | 9.52 | 38 |
| Boar C | 65.2% | 7.40 | 23 |
| Boar D | 57.1% | 7.17 | 21 |

### Example 5

### Zinc Signature Analysis

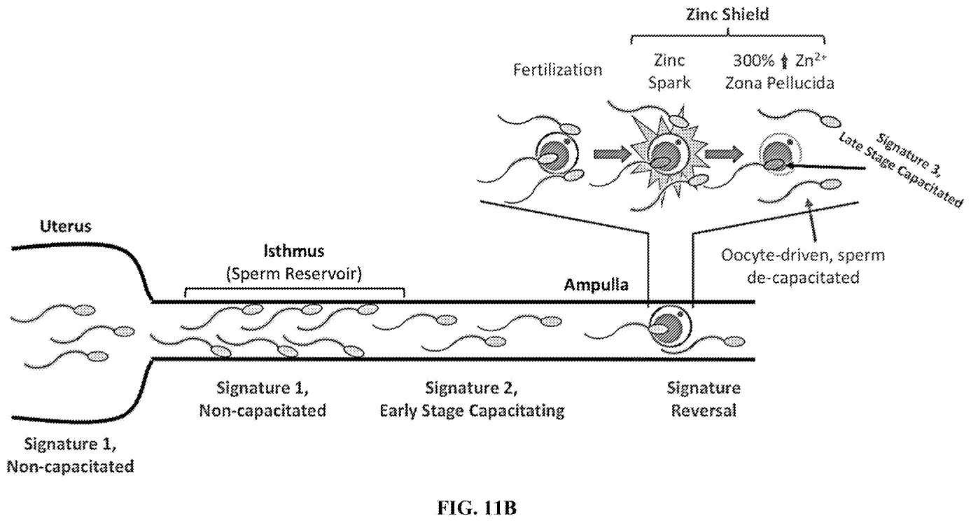

The findings in Examples 1 to 4 are important for livestock and human semen handling methods prior to artificial insemination (AI) or in vitro fertilization. Comparison of zinc signature patterns in boars with varied fertility indicates potential of Zn²⁺ probes in the evaluation of livestock sperm quality. While such findings with a small group of boars are preliminary, Zn²⁺ fluorometry could be also given consideration in human andrology and infertility diagnostics. For example, these findings not only indicate the existence of sperm subpopulations capable/incapable of fertilizing the oocyte, but even more so that sequential capacitation and resulting waves of sperm release from the sperm reservoir, originally thought to be primarily driven by female reproductive tract-issued signals²⁷⁻²⁹, are rather co-dependent of sperm sub-population (FIG. 11A and FIG. 11B). Optimization of semen Zn²⁺ and/or zinc containing protein(s) levels could thus improve the outcomes of AI in livestock and assisted reproductive therapy in humans. Based on the present data, sperm zinc signature likely changes as the spermatozoa advance through the female reproductive tract and progress through different stages of capacitation; the proposed, reciprocal sperm and oocyte Zn²⁺-signaling for the blockage of polyspermy is outlined in FIG. 11B. Such findings shift the paradigm of anti-polyspermy defense mechanisms and demonstrate that sperm zinc signature is an early indicator of sperm capacitation and a candidate biomarker of sperm quality/fertility.

### REFERENCES

1. Que, E. L. et al. Quantitative mapping of zinc fluxes in the mammalian egg reveals the origin of fertilization-induced zinc sparks. Nat Chem 7, 130-139, doi:10.1038/nchem.2133 (2015).
2. Zhang, N., Duncan, F. E., Que, E. L., O'Halloran, T. V. & Woodruff, T. K. The fertilization-induced zinc spark is a novel biomarker of mouse embryo quality and early development. Sci Rep 6, 22772, doi:10.1038/srep22772 (2016).
3. Lishko, P. V., Botchkina, I. L., Fedorenko, A. & Kirichok, Y. Acid extrusion from human spermatozoa is mediated by flagellar voltage-gated proton channel. Cell 140, 327-337, doi:10.1016/j.ce11.2009.12.053 (2010).
4. Tardif, S., Dube, C. & Bailey, J. L. Porcine sperm capacitation and tyrosine kinase activity are dependent on bicarbonate and calcium but protein tyrosine phosphorylation is only associated with calcium. Biology of reproduction 68, 207-213, doi:10.1095/biolreprod.102.005082 (2003).
5. Boerke, A., Tsai, P. S., Garcia-Gil, N., Brewis, I. A. & Gadella, B. M. Capacitation-dependent reorganization of microdomains in the apical sperm head plasma membrane: functional relationship with zona binding and the zona-induced acrosome reaction. Theriogenology 70, 1188-1196, doi:10.1016/j.theriogenology.2008.06.021 (2008).
6. Sutovsky, P. Sperm proteasome and fertilization. Reproduction 142, 1-14, doi:10.1530/REP-11-0041 (2011).
7. Kerns, K., Morales, P. & Sutovsky, P. Regulation of Sperm Capacitation by the 26S Proteasome: An Emerging New Paradigm in Spermatology. Biology of reproduction 94, 117, doi:10.1095/biolreprod.115.136622 (2016).
8. Hillman, P., Ickowicz, D., Vizel, R. & Breitbart, H. Dissociation between AKAP3 and PKARII promotes AKAP3 degradation in sperm capacitation. PloS one 8, e68873, doi:10.1371/journal.pone.0068873 (2013).
9. Yi, Y. J. et al. Ubiquitin-activating enzyme (UBA1) is required for sperm capacitation, acrosomal exocytosis and sperm-egg coat penetration during porcine fertilization. Int J Androl 35, 196-210, doi:10.1111/j.1365-2605.2011.01217.x (2012).
10. Jaiswal, B. S. & Eisenbach, M. in Fertilization (ed Daniel M. Hardy) Ch. 3, 57-117 (Academic Press, 2002).
11. Zimmerman, S. W. et al. Sperm proteasomes degrade sperm receptor on the egg zona pellucida during mammalian fertilization. PloS one 6, e17256, doi:10.1371/journal.pone.0017256 (2011).
12. Yi, Y. J. et al. Sperm-surface ATP in boar spermatozoa is required for fertilization: relevance to sperm proteasomal function. Syst Biol Reprod Med 55, 85-96, doi:10.1080/19396360802699074 (2009).
13. Miles, E. L. et al. Transgenic pig carrying green fluorescent proteasomes. Proc Natl Acad Sci USA 110, 6334-6339, doi:10.1073/pnas.1220910110 (2013).
14. Stoltenberg, M., Sorensen, M. & Danscher, G. Histochemical demonstration of zinc ions in ejaculated human semen. International journal of andrology 20, 229-236, doi:10.1046/j.1365-2605.1997.00060.x (1997).
15. Zimmerman, S. W. et al. Identification and characterization of RING-finger ubiquitin ligase UBR7 in mammalian spermatozoa. Cell Tissue Res 356, 261-278, doi:10.1007/s00441-014-1808-x (2014).
16. Bianchi, F., Rousseaux-Prevost, R., Sautiere, P. & Rousseaux, J. P2 protamines from human sperm are zinc-finger proteins with one CYS2/HIS2 motif. Biochemical and biophysical research communications 182, 540-547, doi:10.1016/0006-291X(92) 91766-J (1992).
17. Nanassy, L., Liu, L., Griffin, J. & Carrell, D. T. The clinical utility of the protamine 1/protamine 2 ratio in sperm. Protein and peptide letters 18, 772-777, doi:10.2174/092986611795713934 (2011).
18. Ambroggio, X. I., Rees, D. C. & Deshaies, R. J. JAMM: A Metalloprotease-Like Zinc Site in the Proteasome and Signalosome. PLOS Biology 2, e2, doi:10.1371/journal.pbio.0020002 (2003).
19. Nagase, H. & Woessner, J. F., Jr. Matrix metalloproteinases. The Journal of biological chemistry 274, 21491-21494, doi:10.1074/jbc.274.31.21491 (1999).
20. Shimokawa Ki, K. et al. Matrix metalloproteinase (MMP)-2 and MMP-9 activities in human seminal plasma. Molecular human reproduction 8, 32-36 (2002).
21. Beek, J., Nauwynck, H., Maes, D. & Van Soom, A. Inhibitors of zinc-dependent metalloproteases hinder sperm passage through the cumulus oophorus during porcine fertilization in vitro. Reproduction 144, 687-697, doi:10.1530/REP-12-0311 (2012).
22. Bjorndahl, L. & Kvist, U. Human sperm chromatin stabilization: a proposed model including zinc bridges. Molecular human reproduction 16, 23-29, doi:10.1093/molehr/gap099 (2010).
23. Kvist, U. Importance of spermatozoal zinc as temporary inhibitor of sperm nuclear chromatin decondensation ability in man. Acta physiologica Scandinavica 109, 79-84, doi:10.1111/j.1748-1716.1980.tb06567.x (1980).
24. Holt, W. V. & North, R. D. Thermotropic phase transitions in the plasma membrane of ram spermatozoa. J Reprod Fertil 78, 447-457, doi:10.1530/jrf.0.0780447 (1986).
25. Qi, H. et al. All four CatSper ion channel proteins are required for male fertility and sperm cell hyperactivated motility. Proc Natl Acad Sci USA 104, 1219-1223, doi:10.1073/pnas.0610286104 (2007).
26. Buffone, M. G., Calamera, J. C., Verstraeten, S. V. & Doncel, G. F. Capacitation-associated protein tyrosine phosphorylation and membrane fluidity changes are impaired in the spermatozoa of asthenozoospermic patients. Reproduction 129, 697-705, doi:10.1530/rep.1.00584 (2005).
27. Suarez, S. S. Regulation of sperm storage and movement in the mammalian oviduct. Int J Dev Biol 52, 455-462, doi:10.1387/ijdb.072527ss (2008).
28. Leemans, B. et al. An alkaline follicular fluid fraction induces capacitation and limited release of oviduct epithelium-bound stallion sperm. Reproduction 150, 193-208, doi:10.1530/rep-15-0178 (2015).
29. Giojalas, L. C., Rovasio, R. A., Fabro, G., Gakamsky, A. & Eisenbach, M. Timing of sperm capacitation appears to be programmed according to egg availability in the female genital tract. Fertility and sterility 82, 247-249, doi:10.1016/j.fertnstert.2003.11.046 (2004).
30. Andreychenko, S. V. et al. Post-Chornobyl remote radiation effects on human sperm and seminal plasma characteristics. Experimental oncology 38, 245-251 (2016).
31. Nenkova, G., Petrov, L. & Alexandrova, A. Role of Trace Elements for Oxidative Status and Quality of Human Sperm. Balkan medical journal, doi:10.4274/balkanmedj.2016.0147 (2017).
32. Colagar, A. H., Marzony, E. T. & Chaichi, M. J. Zinc levels in seminal plasma are associated with sperm quality in fertile and infertile men. Nutrition research (New York, N.Y.) 29, 82-88, doi:10.1016/j.nutres.2008.11.007 (2009).
33. Zhao, J. et al. Zinc levels in seminal plasma and their correlation with male infertility: A systematic review and meta-analysis. Sci Rep 6, doi:10.1038/srep22386 (2016).
34. Harrison, R. A., Dott, H. M. & Foster, G. C. Effect of ionic strength, serum albumin and other macromolecules on the maintenance of motility and the surface of mammalian spermatozoa in a simple medium. J Reprod Fertil 52, 65-73, doi:10.1530/jrf.0.0520065 (1978).
35. Beyersmann, D. & Haase, H. Functions of zinc in signaling, proliferation and differentiation of mammalian cells. Biometals: an international journal on the role of metal ions in biology, biochemistry, and medicine 14, 331-341 (2001).
36. Kim, I. et al. Pyrrolidine dithiocarbamate and zinc inhibit proteasome-dependent proteolysis. Experimental cell research 298, 229-238, doi:10.1016/j.yexcr.2004.04.017 (2004).
37. Massanyi, P. et al. Concentration of copper, iron, zinc, cadmium, lead, and nickel in boar semen and relation to the spermatozoa quality. Journal of environmental science and health. Part A, Toxic/hazardous substances & environmental engineering 38, 2643-2651, doi:10.1081/ESE-120024453 (2003).
38. Mogielnicka-Brzozowska, M., Wysocki, P., Strzezek, J. & Kordan, W. Zinc-binding proteins from boar seminal plasma—isolation, biochemical characteristics and influence on spermatozoa stored at 4 degrees C. Acta biochimica Polonica 58, 171-177 (2011).
39. Henkel, R., Bittner, J., Weber, R., thither, F. & Miska, W. Relevance of zinc in human sperm flagella and its relation to motility. Fertility and sterility 71, 1138-1143, doi:10.1016/S0015-0282(99) 00141-7 (1999).
40. Sorensen, M. B., Stoltenberg, M., Danscher, G. & Ernst, E. Chelation of intracellular zinc ions affects human sperm cell motility. MHR: Basic science of reproductive medicine 5, 338-341, doi:10.1093/molehr/5.4.338 (1999).
41. Que, E. L. et al. Zinc sparks induce physiochemical changes in the egg zona pellucida that prevent polyspermy. Integrative biology: quantitative biosciences from nano to macro 9, 135-144, doi:10.1039/c6ib00212a (2017).
42. Guidobaldi, H. A. et al. Sperm chemorepulsion, a supplementary mechanism to regulate fertilization. Human reproduction 32, 1560-1573, doi:10.1093/humrep/dex232 (2017).
43. Ferrer, M. et al. MMP2 and acrosin are major proteinases associated with the inner acrosomal membrane and may cooperate in sperm penetration of the zona pellucida during fertilization. Cell and Tissue Research 349, 881-895, doi:10.1007/s00441-012-1429-1 (2012).
44. Steven, F. S., Griffin, M. M. & Chantler, E. N. Inhibition of human and bovine sperm acrosin by divalent metal ions. Possible role of zinc as a regulator of acrosin activity. Int J Androl 5, 401-412, doi:10.1111/j.1365-2605.1982.tb00270.x (1982).
45. Ciereszko, A., Dabrowski, K., Mims, S. D. & Glogowski, J. Characteristics of sperm acrosin-like activity of paddlefish (Polyodon spathula Walbaum). Comparative biochemistry and physiology. Part B, Biochemistry & molecular biology 125, 197-203, doi:10.1016/S0305-0491(99) 00167-4 (2000).
46. Backstrom, J. R., Miller, C. A. & Tokes, Z. A. Characterization of neutral proteinases from Alzheimer-affected and control brain specimens: identification of calcium-dependent metalloproteinases from the hippocampus. Journal of neurochemistry 58, 983-992, doi:10.1111/j.1471-4159.1992.tb09352.x (1992).
47. Stephenson, J. L. & Brackett, B. G. Influences of zinc on fertilisation and development of bovine oocytes in vitro. Zygote 7, 195-201, doi:10.1017/5096719949900057x (1999).
48. Noguchi, S. & Nakano, M. Structure of the acidic N-linked carbohydrate chains of the 55-kDa glycoprotein family (PZP3) from porcine zona pellucida. European Journal of Biochemistry 209, 883-894, doi:10.1111/j.1432-1033.1992.tb17361.x (1992).
49. Yurewicz, E. C., Pack, B. A. & Sacco, A. G. Isolation, composition, and biological activity of sugar chains of porcine oocyte zona pellucida 55K glycoproteins. Mol Reprod Dev 30, 126-134, doi:10.1002/mrd.1080300209 (1991).
50. Noguchi, S., Hatanaka, Y., Tobita, T. & Nakano, M. Structural analysis of the N-linked carbohydrate chains of the 55-kDa glycoprotein family (PZP3) from porcine zona pellucida. European journal of biochemistry/FEBS 204, 1089-1100, doi:doi.org/10.1111/j.1432-1033.1992.tb16733.x (1992).
51. Amari, S. et al. Essential role of the nonreducing terminal alpha-mannosyl residues of the N-linked carbohydrate chain of bovine zona pellucida glycoproteins in sperm-egg binding. Mol Reprod Dev 59, 221-226, doi:10.1002/mrd.1026 (2001).
52. Yi, Y. J. et al. Ubiquitin-activating enzyme (UBA1) is required for sperm capacitation, acrosomal exocytosis and sperm-egg coat penetration during porcine fertilization. International Journal of Andrology 35, 196-210, doi:10.1111/j.1365-2605.2011.01217.x (2012).
53. Flesch, F. M., Colenbrander, B., van Golde, L. M. G. & Gadella, B. M. Capacitation Induces Tyrosine Phosphorylation of Proteins in the Boar Sperm Plasma Membrane. Biochemical and biophysical research communications 262, 787-792, doi:10.1006/bbrc.1999.1300 (1999).
54. Tardif, S., Dube, C., Chevalier, S. & Bailey, J. L. Capacitation is associated with tyrosine phosphorylation and tyrosine kinase-like activity of pig sperm proteins. Biology of reproduction 65, 784-792, doi:10.1095/biolreprod65.3.784 (2001).
55. Dube, C., Tardif, S., LeClerc, P. & Bailey, J. L. The importance of calcium in the appearance of p32, a boar sperm tyrosine phosphoprotein, during in vitro capacitation. J Androl 24, 727-733 (2003).
56. Kennedy, C. E. et al. Protein expression pattern of PAWP in bull spermatozoa is associated with sperm quality and fertility following artificial insemination. Mol Reprod Dev 81, 436-449, doi:10.1002/mrd.22309 (2014).
40. Duan D, et al. Standard operating procedures (SOPs) for evaluating the heart in preclinical studies of Duchenne muscular dystrophy. J Cardiovasc Transl Res. 2016; 9(1):85-86.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawings shall be interpreted as illustrative and not in a limiting sense.

We claim:

1. A method for determining fertility of spermatozoa in a sperm sample obtained from a sperm source or for determining fertility of the sperm source comprising:
labeling the sperm sample with a zinc probe,
identifying presence and/or localization of zinc associated with the spermatozoa in the sample;
comparing the presence and/or localization of the zinc associated with the spermatozoa to a reference pattern of zinc presence and/or localization associated with sperm capacitation to determine whether or not the spermatozoa in the sample or the source of the sperm sample is fertile.

2. The method of claim 1 wherein the sperm sample is determined to comprise fertile spermatozoa when spermatozoa are identified having a zinc signature 1 and/or a zinc signature 2 and/or the sample is determined to comprise infertile spermatozoa when spermatozoa are identified having a zinc signature 3 and/or a zinc signature 4, wherein:
the zinc signature 1 is characterized by zinc presence and/or localization of the zinc in the sperm head and whole tail;
the zinc signature 2 is characterized by zinc presence and/or localization of the zinc in both the sperm head and tail midpiece, excluding the tail principal piece;
the zinc signature 3 is characterized by localization of zinc only in the sperm tail midpiece;
and the zinc signature 4 is characterized by essentially no zinc presence in the spermatozoa.

3. The method of claim 2 wherein the zinc signature 1 is associated with non-capacitated spermatozoa.

4. The method of claim 2 wherein the zinc signature 2 is associated with hyperactivated spermatozoa capable of recognizing and binding an oocyte zona pellucida.

5. The method of claim 2 wherein the zinc signature 3 is associated with spermatozoa that are undergoing or have fully underwent capacitation, have undergone acrosomal modification, and/or have a compromised/remodeled plasma membrane.

6. The method of claim 2 wherein the zinc signature 4 is associated with spermatozoa having a compromised/remodeled plasma membrane and an exocytosed acrosome.

7. The method of claim 1 further comprising: labeling the sample with a compound for detecting a disrupted plasma membrane and/or a compound for detecting modifications to an acrosome; and determining plasma membrane disruption and/or acrosomal modifications in the sample.

8. The method of claim 7 wherein determining plasma membrane disruption and/or acrosomal modifications in the sample comprises comparing the plasma membrane disruption and acrosomal modifications in the sample to plasma membrane disruption and/or acrosomal modifications in (a) a non-capacitated sperm sample, (b) a post capacitated sperm sample.

9. (canceled)

10. The method of claim 8 wherein high fertility sperm and/or sperm associated with signatures 1 and 2 are characterized by reduced plasma membrane disruption and reduced acrosomal modifications and low fertility sperm and/or sperm associated with signatures 3 and 4 are characterized by increased plasma membrane disruption and increased acrosomal modifications.

11. The method of claim 8 wherein the compound for detecting plasma membrane disruption comprises a DNA stain and wherein plasma membrane disruption is detected when nuclei of the spermatozoa are visible.

12. (canceled)

13. The method of claim 8 wherein the compound for detecting modification in the acrosome bind to the outer acrosomal membrane of compromised and/or modified sperm acrosomes and wherein detection of acrosomal modification comprises increased labeling of the compound.

14-15. (canceled)

16. The method of claim 1 further comprising adding exogenous zinc ions to a sample of fertile, subfertile, and/or infertile spermatozoa.

17. (canceled)

18. The method of claim 2 wherein determining the fertility of a sperm source that provided the sperm sample, further comprises exposing the sperm sample or a second sperm sample from the sperm source to in vitro capacitation conditions wherein the sperm source is identified as a fertile sperm source when it achieves an increased subpopulation of spermatozoa having the zinc signature 3 after in vitro capacitation as compared to a sample from the sperm source not put through in vitro capacitation or as compared to a reference pattern.

19-20. (canceled)

21. The method of claim 1, further comprising selecting a fertile sperm source for further use in artificial insemination, in vitro fertilization, and/or breeding.

22. (canceled)

23. The method of claim 1 wherein the sperm source is an in vitro source or a mammalian source.

24. (canceled)

25. The method of claim 1 wherein the zinc presence and/or localization is identified using flow cytometry, microscopy or combinations thereof.

26. The method of claim 25 wherein the flow cytometry comprises image-based flow cytometry.

27. (canceled)

28. The method of claim 25 wherein using flow cytometry comprises gating and/or masking of the flow cytometry data.

29-34. (canceled)

35. A method of improving fertility of a sperm sample, the method comprising adding exogenous zinc ions to the sample.

36. (canceled)

37. A kit for determining the fertility of spermatozoa comprising:
a zinc probe and one or more of (a) a compound for detecting plasma membrane integrity, (b) a compound for detecting modifications to the acrosome, and (c) a DNA dye.

38-48. (canceled)
